(19) United States
(12) Patent Application Publication
(10) Pub. No.: US 2025/0304999 A1
(43) Pub. Date: Oct. 2, 2025

(54) GENETICALLY MODIFIED ENTEROVIRUS VECTORS WITH ENHANCED GENOMIC STABILITY

(71) Applicant: VIROGIN BIOTECH CANADA LTD, Richmond (CA)

(72) Inventors: Honglin Luo, Vancouver (CA); William Wei-Guo Jia, Vancouver (CA); Huitao Liu, Vancouver (CA)

(21) Appl. No.: 18/866,006

(22) PCT Filed: May 19, 2023
(86) PCT No.: PCT/US2023/023012

§ 371 Date: Nov. 14, 2024

Related U.S. Application Data
(60) Provisional application No. 63/344,316, filed on May 20, 2022.

(51) Int. Cl. C12N 15/86; A61K 35/768; A61P 35/00

(52) U.S. Cl. CPC C12N 15/86; A61K 35/768; A61P 35/00; C12N 2770/32332; C12N 2770/32343

(57) ABSTRACT

A replicating oncolytic virus vector is provided having a modified Enterovirus genome (e.g., a Poliovirus, Coxsackievirus or Echovirus genome), wherein the modified Enterovirus genome has one or more copies of one or more miRNA target sequences inserted into the UTR region (e.g., via substitution) and/or in-frame within the coding region of the Enterovirus genome. Also provided are compositions and methods for treating cancer (including for example, lung cancer).

### INCORPORATION BY REFERENCE TO ANY PRIORITY APPLICATIONS

Any and all applications for which a foreign or domestic priority claim is identified in the Application Data Sheet as filed with the present application are hereby incorporated by reference.

### FIELD OF THE INVENTION

The present invention relates generally to genetically modified oncolytic Enterovirus vectors and uses thereof which have reduced toxicity in normal tissues and enhanced genomic stability.

### BACKGROUND

Cancer includes a wide variety of diseases that involve the uncontrolled or abnormal growth of cells that spread or invade into other tissues of the body, initially resulting in changes in bodily function (depending on the type of cancer), and ultimately in death. About 14.1 million new cases of cancer occur each year (excluding skin cancer other than melanoma).

In much of the Western world, lung cancer is the 3rd and 2nd most common cancer for men and women, respectively, and the leading cause of cancer-related deaths for both sexes. Non-small-cell lung cancer (“NSCLC”) constitutes ˜85% of lung cancer cases. Among them, adenocarcinoma is the most common type of lung cancer, accounting for almost half of all lung cancers. Genetic mutations play critical roles in the development of NSCLC. Well-identified oncogenic driver mutations include epidermal growth factor receptor (“EGFR”) and Kirsten rat sarcoma viral oncogene homolog (“KRAS”), which occur in ˜15% and ˜30% of lung adenocarcinoma, respectively Although EGFR mutations can be clinically targeted, KRAS mutations are currently very difficult to treat and associated with a poor prognosis. Small-cell lung cancer (“SCLC”) accounts for ˜15% of all lung cancers. Between 60% to 90% of SCLC cases are featured by mutations in gene encoding tumor protein p53 and/or retinoblastoma protein (Rb). There is also no targeted therapy for SCLC.

A number of therapies have been developed to treat cancer, including for example, radiation therapy, chemotherapy, surgical removal of the cancer, or some combination of these therapies. One new area of therapy that has shown progress is ‘targeted therapy,’ wherein compositions and methods are used to specifically target and kill tumor cells (as opposed to ‘normal’ cells).

One example of a targeted therapy are oncolytic viruses. Briefly, an oncolytic virus is defined as one that is capable of inducing lysis of tumor cells via its self-replication process, and preferably, without causing substantive damage to normal tissues. The greatest advantage of oncolytic viruses over other cancer therapies is that the candidate viruses can be genetically manipulated to increase their potency against specific cancer types. In 2015, the FDA approved the first genetically modified herpes simplex virus 1 (Talimogene laherparepvec or “T-VEC”) for the treatment of melanoma. Over the past decades, several different oncolytic viruses, including adenovirus, measles virus, Newcastle disease virus, retrovirus and vaccinia virus have all been tested in clinical trials for the treatment of cancer. Other viruses, such as coxsackievirus have been suggested for various purposes (see e.g., Kim, DS., Kim, H., Shim, SH. et al. Coxsackievirus B3 used as a gene therapy vector to express functional FGF2. Gene Ther 19, 1159-1165 (2012). https://doi.org/10.1038/gt.2011.201), but have yet to achieve commercial success. The overall anti-cancer efficacy and specificity of many viral vectors remains low, and there is still no FDA-approved virotherapy for lung cancer.

Hence, there remains a need for improved targeted treatments for cancer, e.g., lung cancer, that lyse and destroy transformed cells, while not causing substantive damage to healthy, untransformed cells, and which overcome one or more of the shortcomings associated with the prior art.

All of the subject matter discussed in the Background section is not necessarily prior art and should not be assumed to be prior art merely as a result of its discussion in the Background section. Along these lines, any recognition of problems in the prior art discussed in the Background section or associated with such subject matter should not be treated as prior art unless expressly stated to be prior art. Instead, the discussion of any subject matter in the Background section should be treated as part of the inventor's approach to the particular problem, which in and of itself may also be inventive.

### SUMMARY

Briefly stated, the invention relates to micro-RNA (“miRNA”) based approaches to modify an Enterovirus genome (e.g., a Coxsackievirus such as B3) in order to further enhance its tumor-specificity while reducing off-target toxicity and increasing genome stability to ameliorate the loss of exogenous microRNA target sequences over time.

In one aspect, the invention provides a replicating oncolytic virus vector (i.e., a recombinant vector) having a modified Enterovirus genome (e.g., a Poliovirus, Coxsackievirus or Echovirus genome), wherein the modified Enterovirus genome (e.g., a Poliovirus, Coxsackievirus or Echovirus genome) includes one or more copies of one or more miRNA target sequences. Within preferred embodiments the miRNA target sequences are inserted into the UTR region (e.g., via substitution) and/or in-frame into the coding region of the Enterovirus genome (e.g., a Poliovirus, Coxsackievirus or Echovirus genome). In some embodiments, the Coxsackievirus is Coxsackievirus A or Coxsackievirus B. Within certain embodiments the Enterovirus is Coxsackievirus B3. In other embodiments, the coding region is the P1, P2, or P3 coding region of the Coxsackievirus genome. In further embodiments, the miRNA target sequences are inserted into the UTR region (e.g., via substitution) and/or in-frame between the coding regions for two or more individual genes within the Coxsackievirus genome (e. g. between the genes encoding proteins VP4 and VP2, VP2 and VP3, VP3 and VP1, VP1 and 2A, 2A and 2B, 2B and 2C, 2C and 3A, 3A and 3B, 3B and 3C, and/or 3C and 3D).

In some embodiments, the one or more copies of the one or more_miRNA target sequences include one or more copies of two or more different miRNA target sequences. In other embodiments, the one or more copies of two or more different miRNA target sequences include one or more copies of three or more different miRNA target sequences. In yet other embodiments, the one or more copies of three or more different miRNA target sequences include one or more copies of four or more different miRNA target sequences. In further embodiments, the two or more different miRNA target sequences are targeted by an miRNA selected the group consisting of miR-1, miR-7, miR-30c, miR-124, miR-124*, miR-127, miR-128, miR-129, miR-129*, miR-133, miR-135b, miR-136, miR-136*, miR-137, miR-139-5p, miR-143, miR-154, miR-184, miR-188, miR-204, miR-208, miR-216, miR-217, miR-299, miR-300-3p, miR-300-5p, miR-323, miR-329, miR-337, miR-335, miR-341, miR-369-3p, miR-369-5p, miR-375, miR-376a, miR-376a*, miR-376b-3p, miR-376b-5p, miR-376c, miR-377, miR-379, miR-379*, miR-382, miR-382*, miR-409-5p, miR-410, miR-411, miR-431, miR-433, miR-434, miR-451, miR-466b, miR-485, miR-495, miR-499, miR-539, miR-541, miR-543*, miR-551b, miR-758, and miR-873. By convention, the strand that is more frequently found to be the final product is referred to as miRNA and the rarer partner as miRNA*. In other embodiments, the two or more different miRNA target sequences include target sequences for miR-1, miR-133, miR-216, and miR-375. In yet other embodiments, the two or more different miRNA target sequences include one copy of each miRNA target sequence. In other embodiments, one or more copies of the one or more miRNA target sequences is in a forward orientation and/or one or more copies of the one or more miRNA target sequences is in a reverse orientation. In some embodiments, the recombinant vector further includes at least one nucleic acid encoding a non-viral protein selected from the group consisting of immunostimulatory factors, antibodies, and checkpoint blocking peptides, wherein the at least one nucleic acid is operably linked to a suitable tumor-specific regulatory region. In other embodiments, the non-viral protein is selected from the group consisting of IL12, IL15, IL15 receptor alpha subunit, OX40L, and a PD-L1 blocker.

In another aspect, the invention provides a method for lysing tumor cells, comprising providing an effective amount of a replicating oncolytic virus vector of any of the above embodiments to tumor cells. In some embodiments, the tumor cells include lung cancer cells. In other embodiments the tumor cells include pancreatic cancer cells.

In other aspects, the invention provides a therapeutic composition including at least one replicating oncolytic virus vector of any of the above embodiments and a pharmaceutically acceptable carrier.

In other aspects, the invention provides a method for treating cancer in a subject suffering therefrom, including the step of administering a first composition comprising a therapeutically effective amount of the composition of any of the above embodiments. In some embodiments, the cancer is non-small-cell lung cancer (NSCLC) or small-cell lung cancer (SCLC). In other embodiments, the administration is intravenous (IV) administration, intraperitoneal (IP) administration, or intratumoral (IT) administration.

This Brief Summary has been provided to introduce certain concepts in a simplified form that are further described in detail below in the Detailed Description. Except where otherwise expressly stated, this Brief Summary is not intended to identify key or essential features of the claimed subject matter, nor is it intended to limit the scope of the claimed subject matter.

The details of one or more embodiments are set forth in the description below. The features illustrated or described in connection with one exemplary embodiment may be combined with the features of other embodiments. Thus, any of the various embodiments described herein can be combined to provide further embodiments. Aspects of the embodiments can be modified, if necessary to employ concepts of the various patents, applications and publications as identified herein to provide yet further embodiments. Other features, objects and advantages will be apparent from the description, the drawings, and the claims.

### BRIEF DESCRIPTION OF THE DRAWINGS

Exemplary features of the present disclosure, its nature and various advantages will be apparent from the accompanying drawings and the following detailed description of various embodiments. Non-limiting and non-exhaustive embodiments are described with reference to the accompanying drawings, wherein like labels or reference numbers refer to like parts throughout the various views unless otherwise specified. The sizes and relative positions of elements in the drawings are not necessarily drawn to scale. For example, the shapes of various elements are selected, enlarged, and positioned to improve drawing legibility. The particular shapes of the elements as drawn have been selected for ease of recognition in the drawings. One or more embodiments are described hereinafter with reference to the accompanying drawings in which:

FIGS. 1A, 1B, 1C, and 1D show the relative expression levels of miRNAs in mouse and tumor tissues.

FIG. 2 is a schematic illustration depicting construction of three different embodiments of a miRNA-modified oncolytic Coxsackievirus B3 (“CVB3”) genome.

FIG. 3A is a schematic illustration of primer binding locations relative to the miRNA target sites and genomic organization of three different embodiments of a miRNA- modified oncolytic Coxsackievirus B3 (“CVB3”) genome.

FIGS. 3B, 3C, and 3D are graphs showing viral RNA copy number after serial passaging in cell lines infected with oncolytic CVB3 viruses.

FIGS. 4A and 4B are graphs depicting cell viability data from cell lines infected with oncolytic CVB3 viruses.

FIGS. 5A, 5B, and 5C are graphs showing fold change in viral genome copy numbers in cell lines infected with oncolytic CVB3 viruses.

FIGS. 6A, 6B, 6C, 6D, 6E, and 6F are weight change plots, survival rate plots, histological photographs, and graphs depicting data from mouse model systems (A/J mice) treated with oncolytic CVB3 viruses.

FIGS. 7A, 7B, 7C, 7D, 7E, and 7F are weight change plots, survival rate plots, and graphs depicting data from mouse model systems (SCID mice) treated with oncolytic CVB3 viruses.

FIGS. 8A-8Z, 8AA-8ZZ, and 8AAA-8SSS are a selected list of microRNAs in tumors, all of which are incorporated by reference in their entirety.

### DETAILED DESCRIPTION OF THE INVENTION

The present invention may be understood more readily by reference to the following detailed description of preferred embodiments of the invention and the Examples included herein.

Prior to setting forth the invention in more detail however, it may be helpful to an understanding thereof to first set forth definitions of certain terms that are used hereinafter.

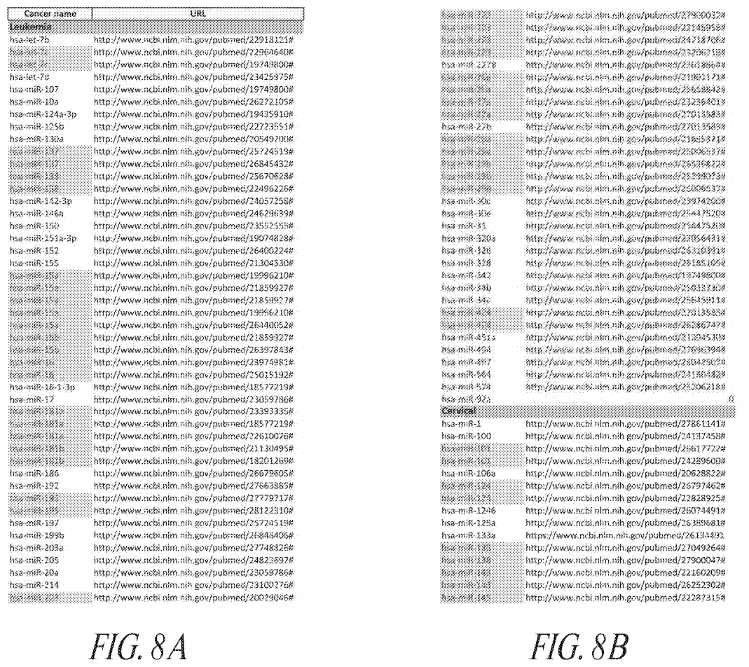

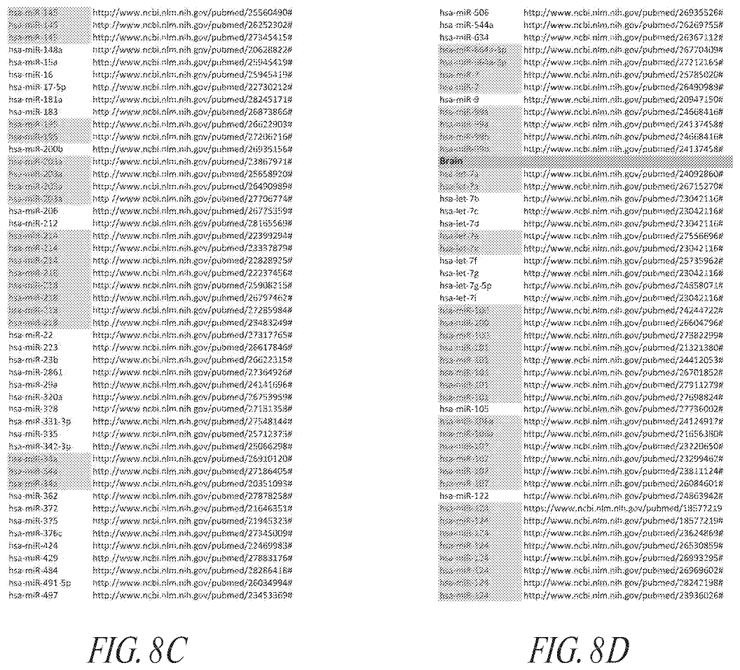

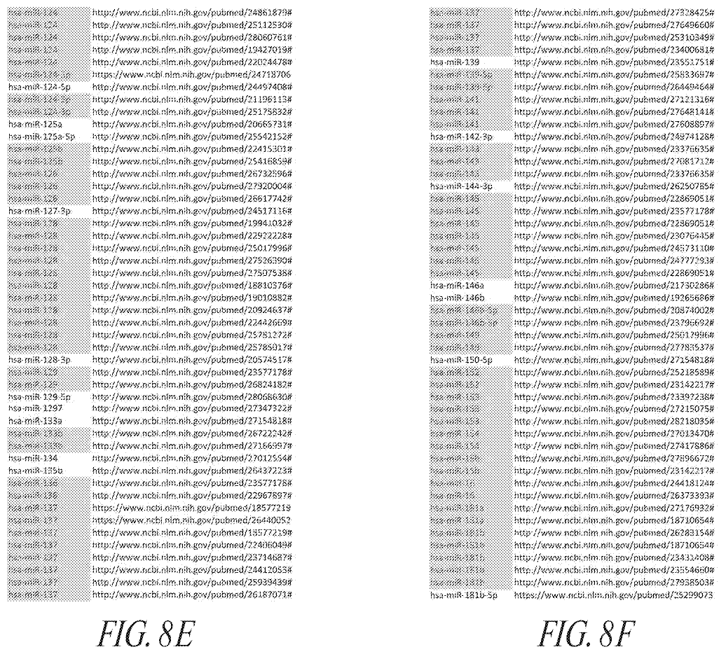

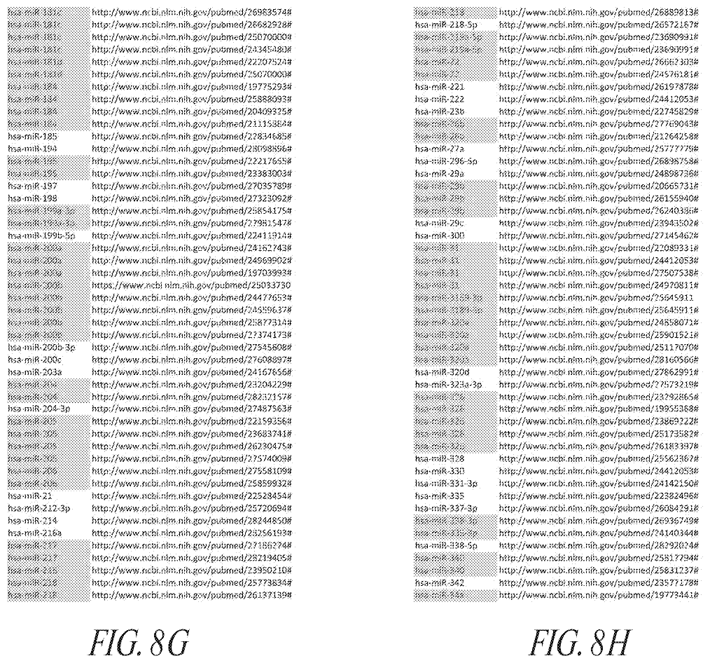

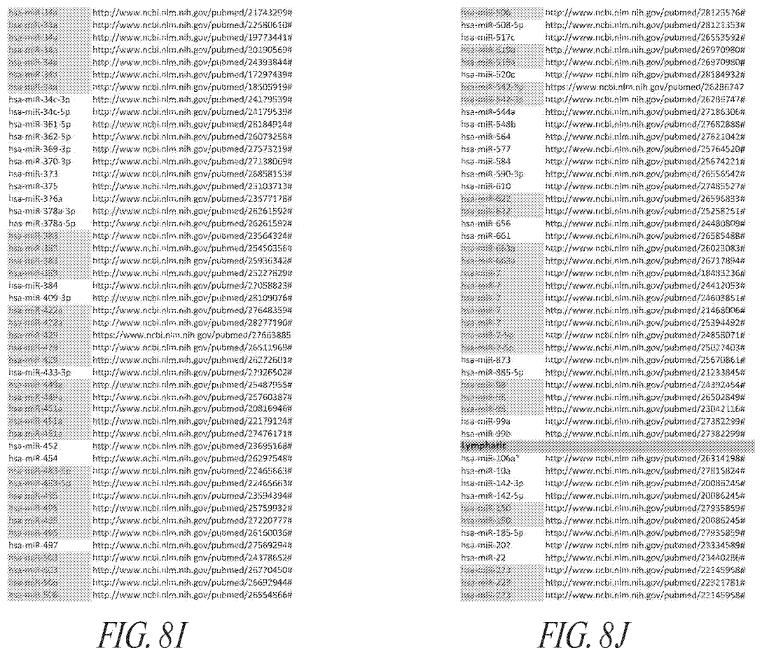

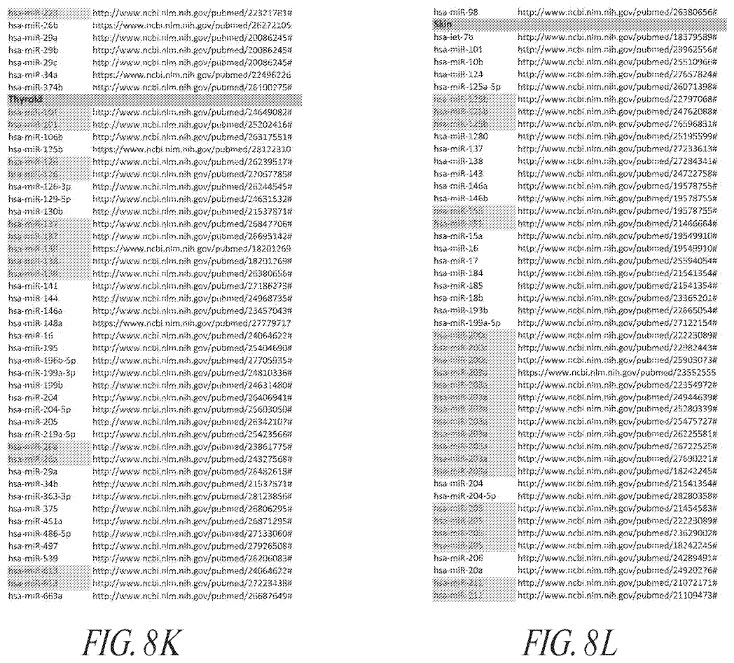

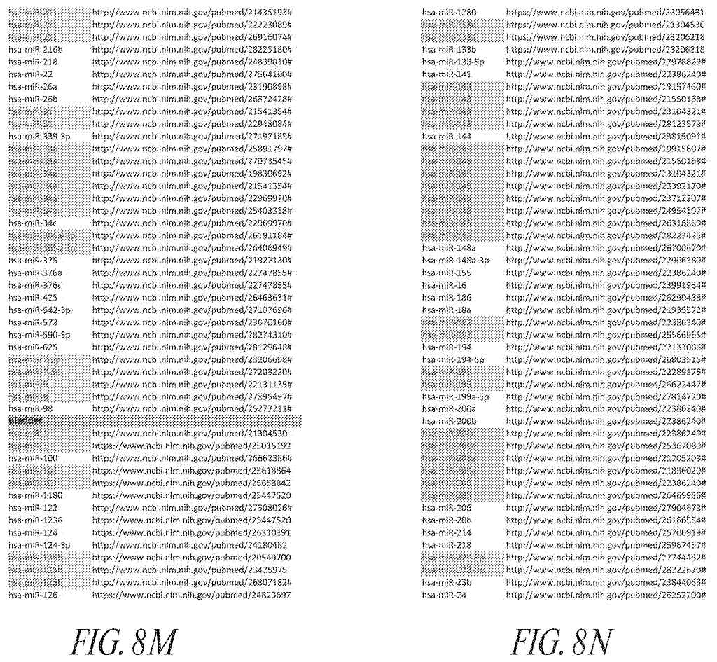

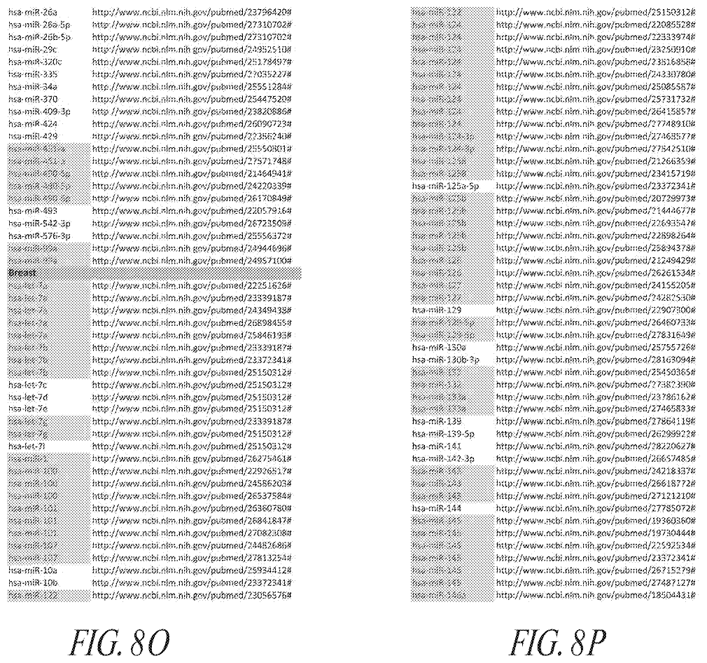

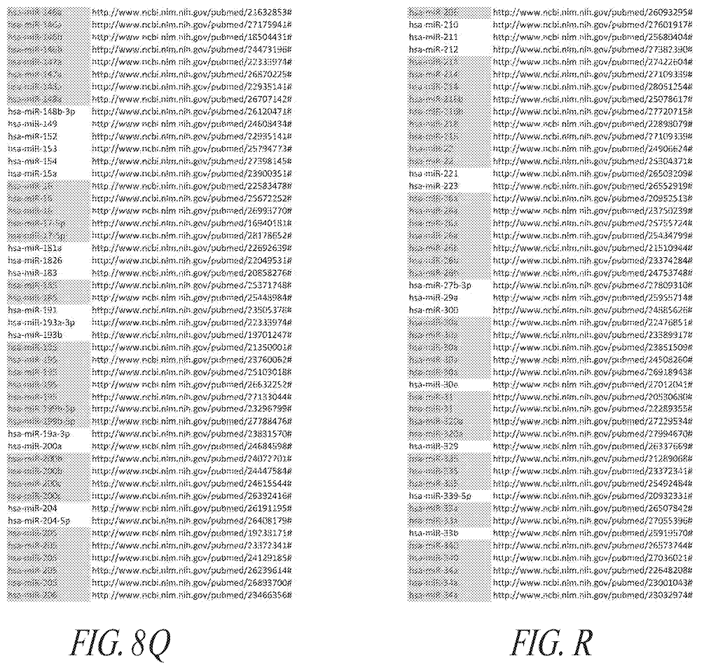

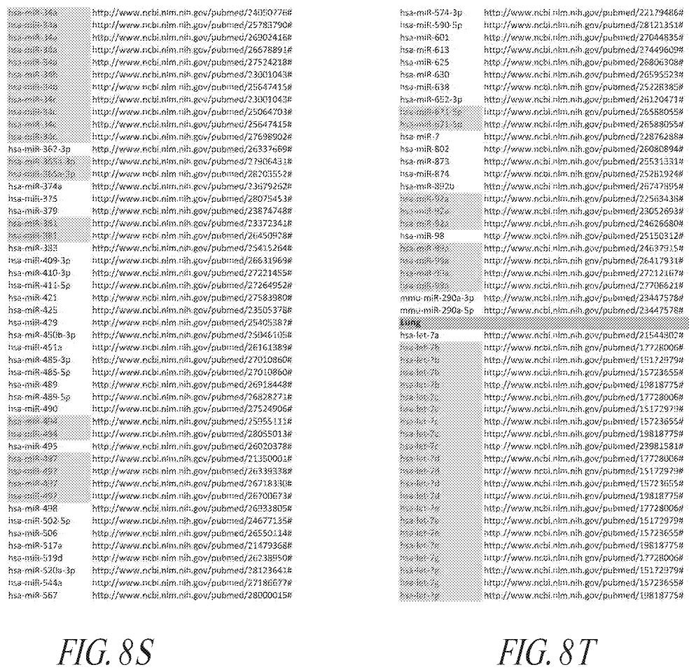

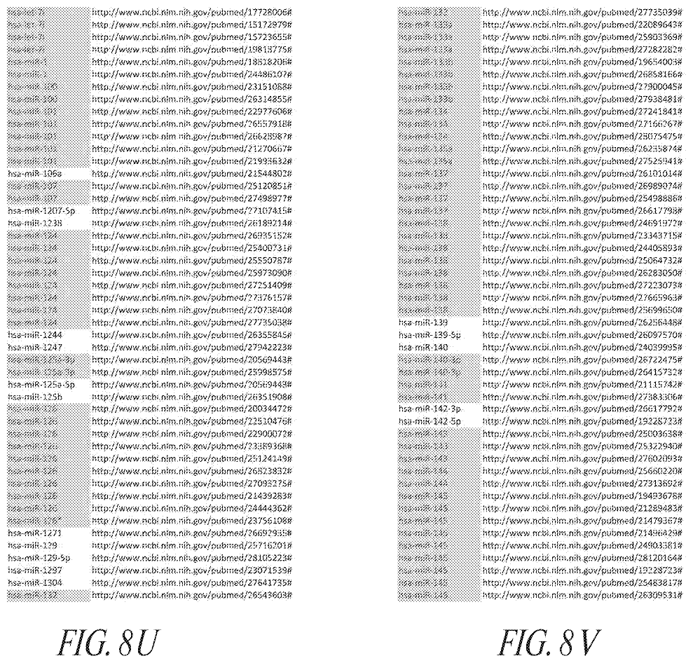

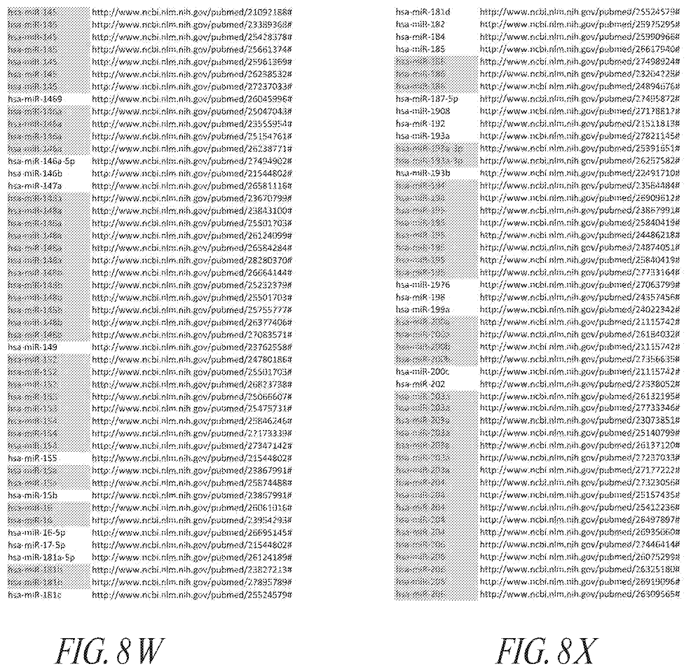

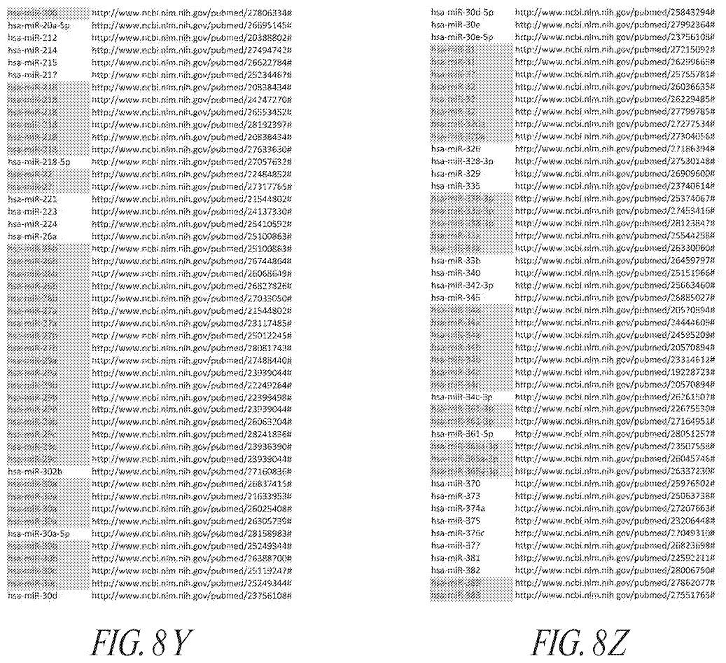

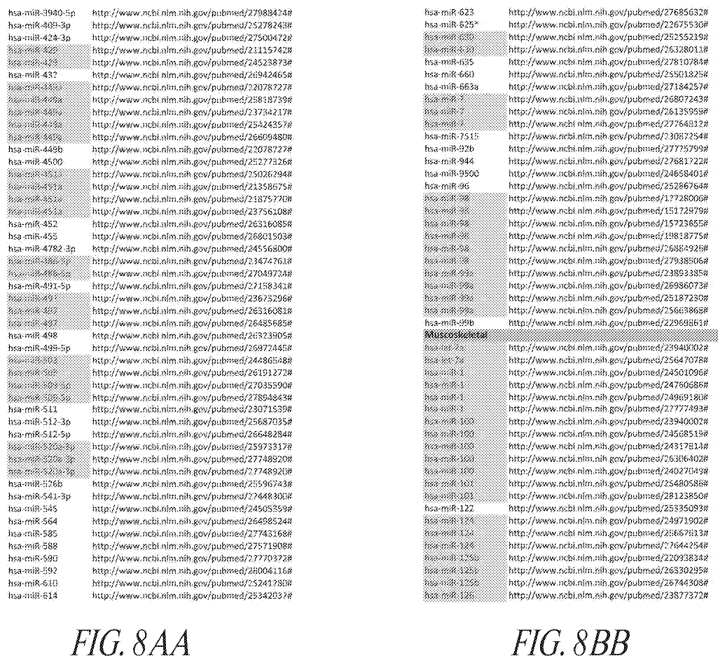

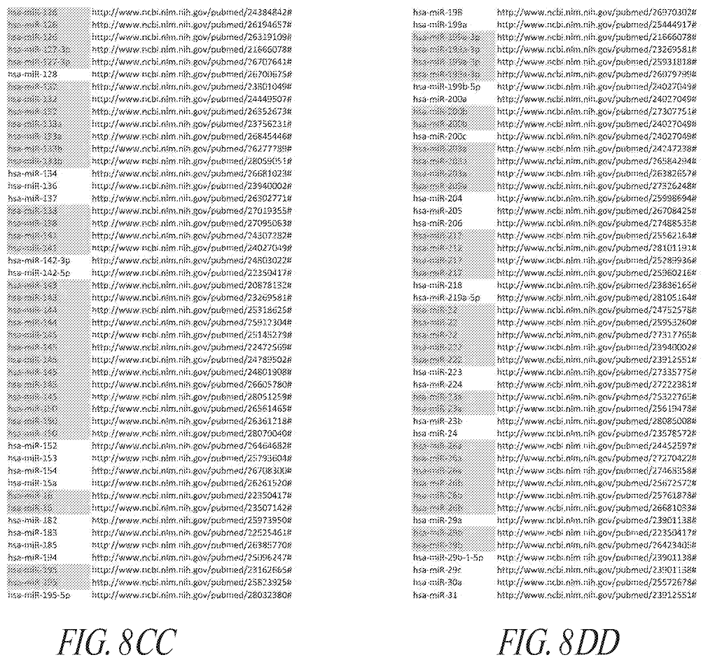

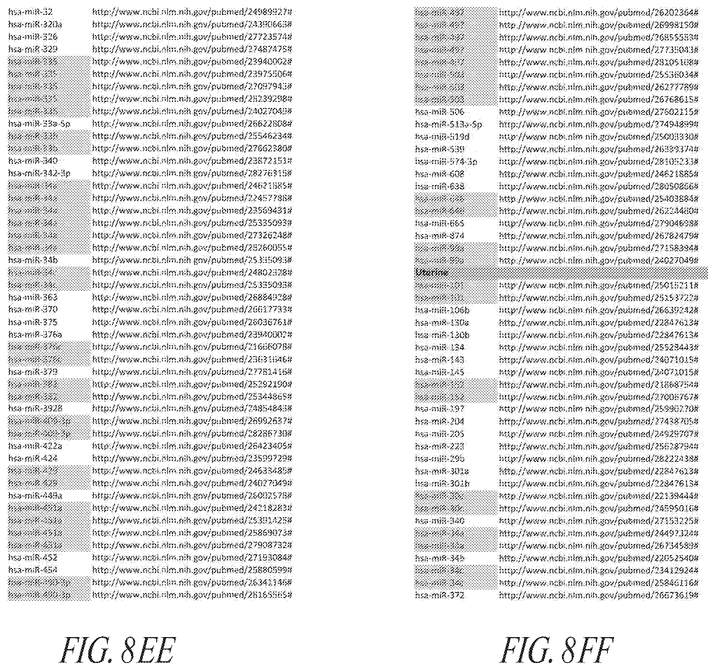

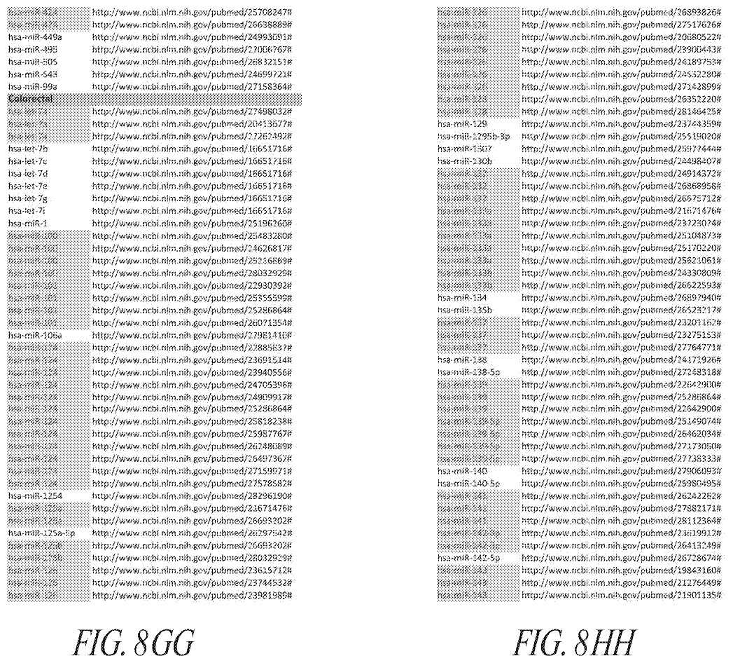

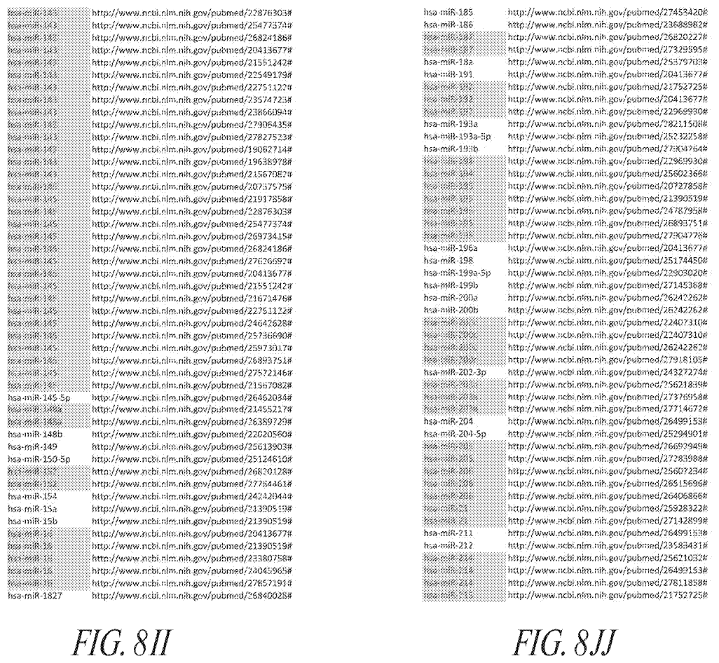

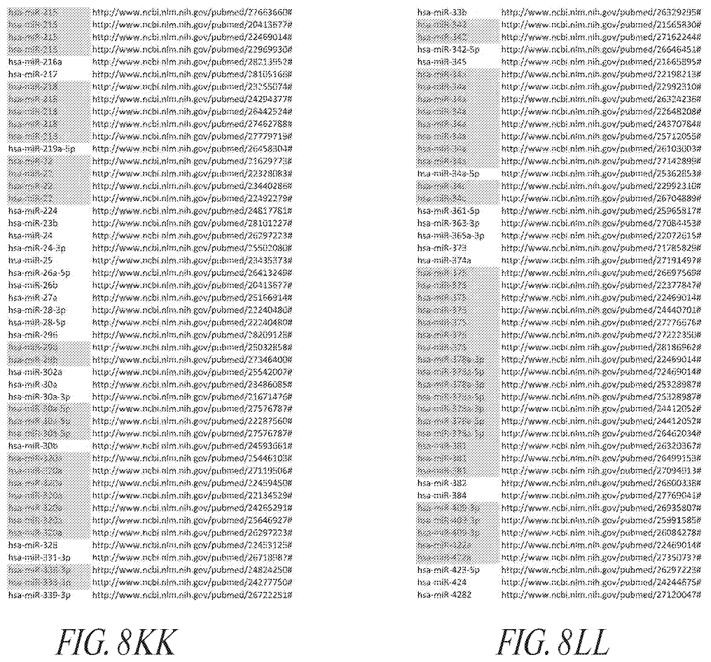

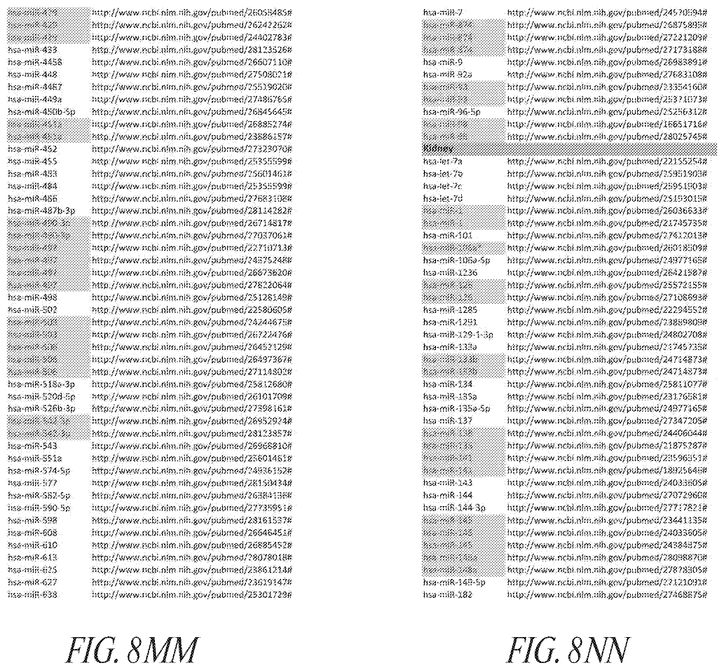

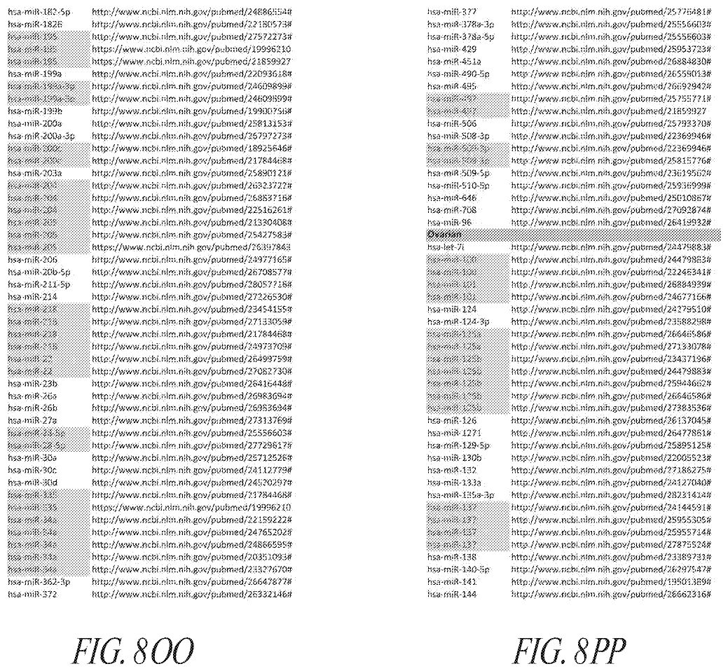

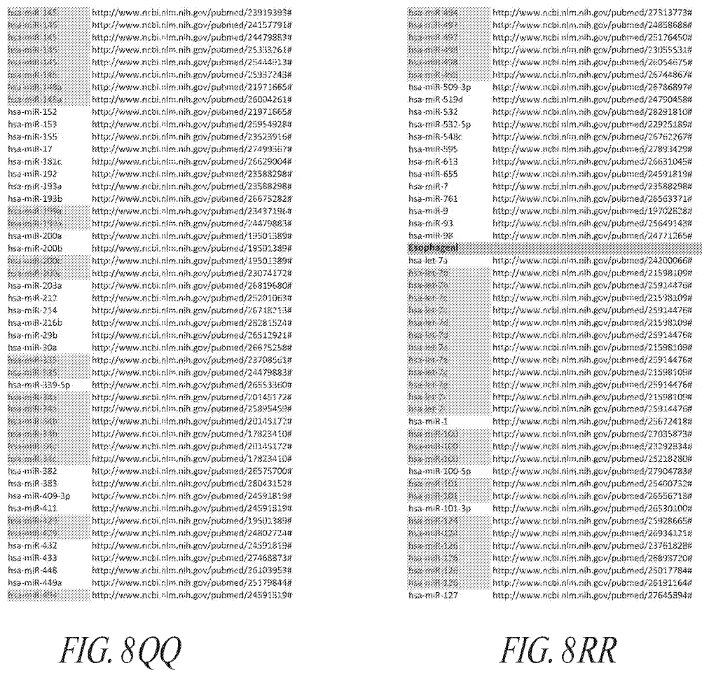

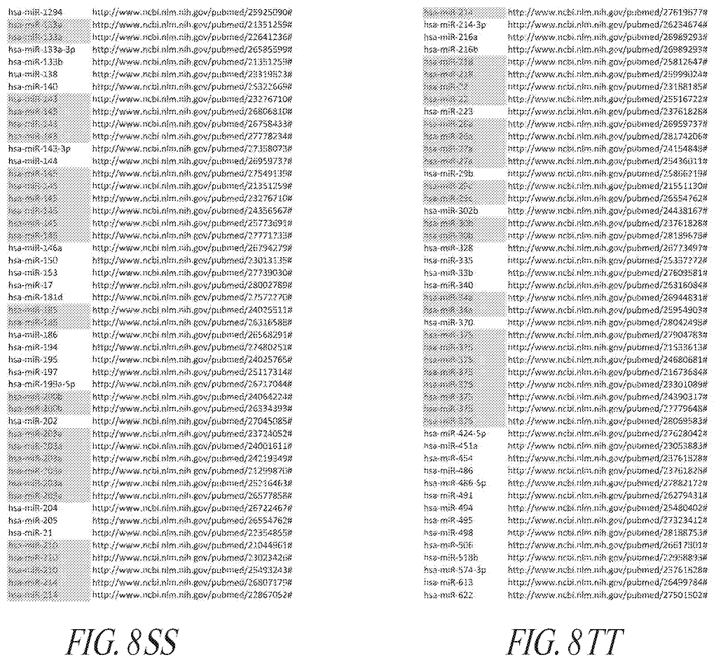

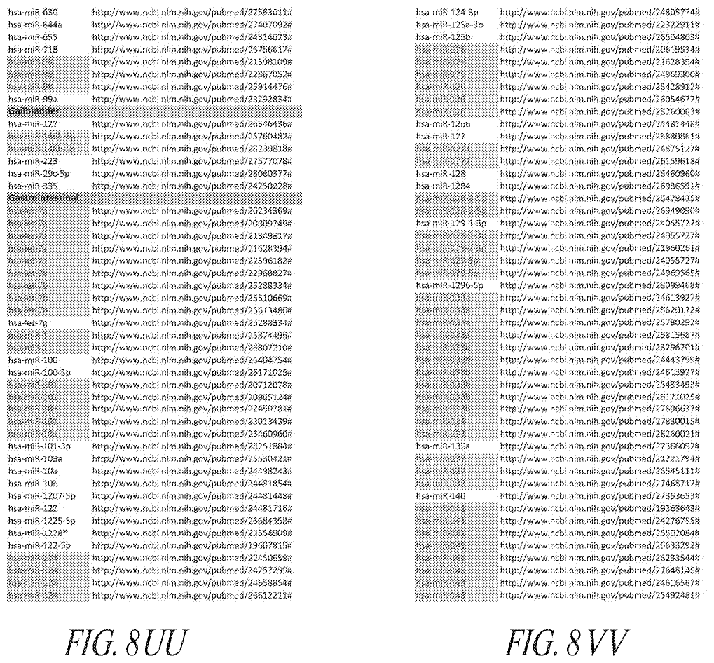

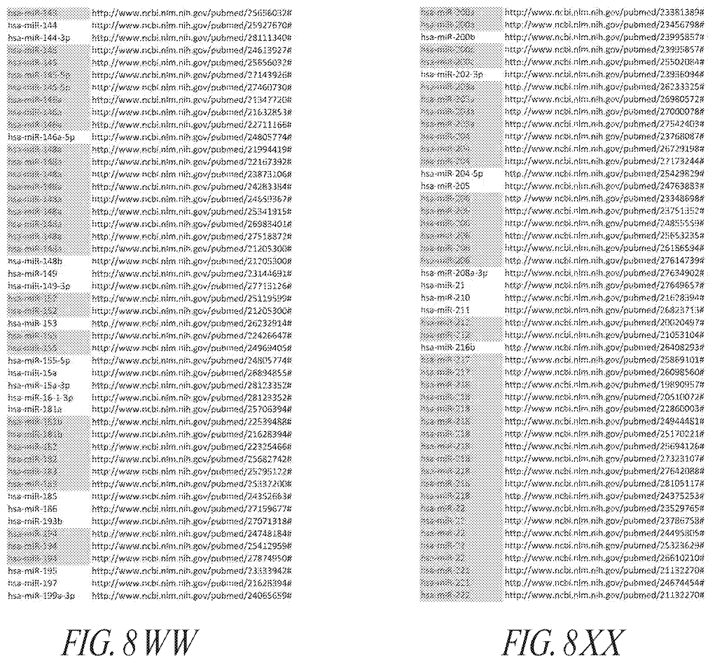

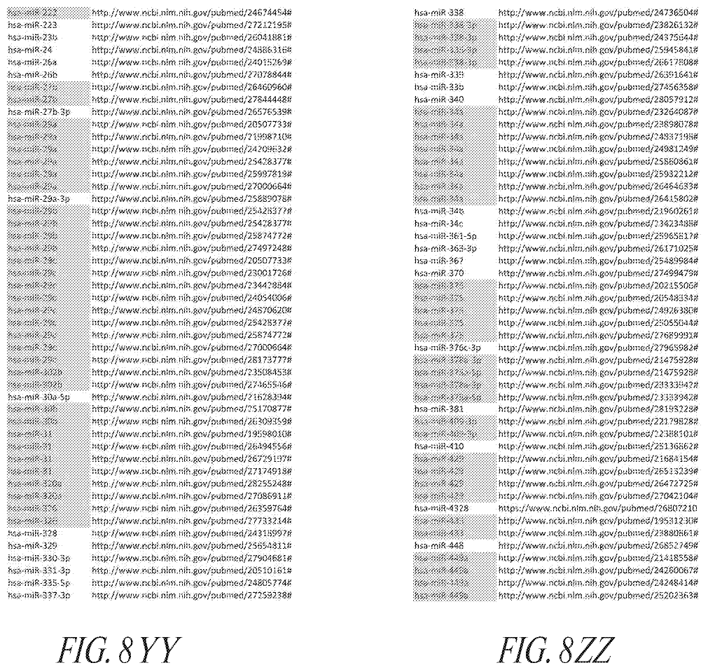

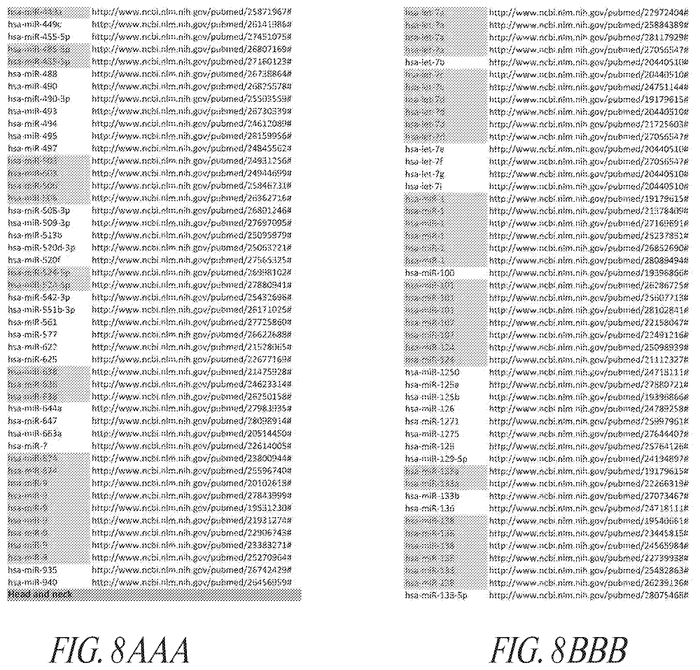

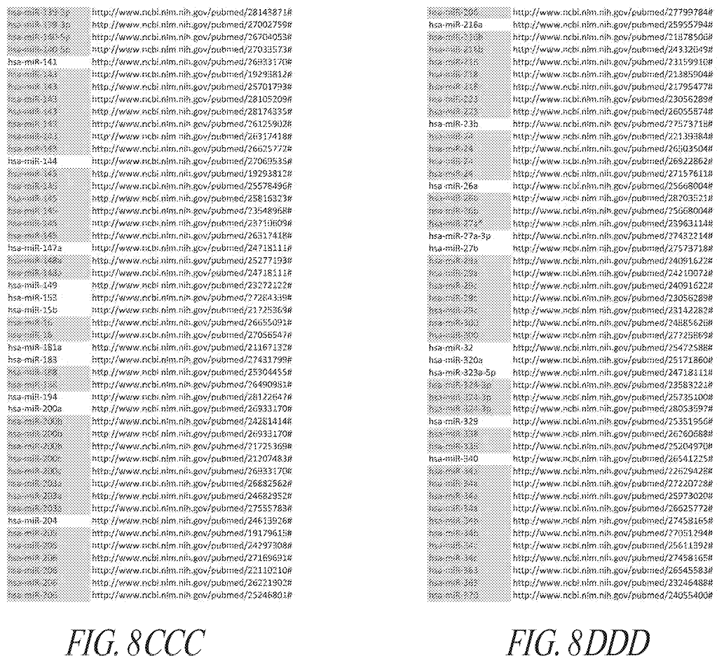

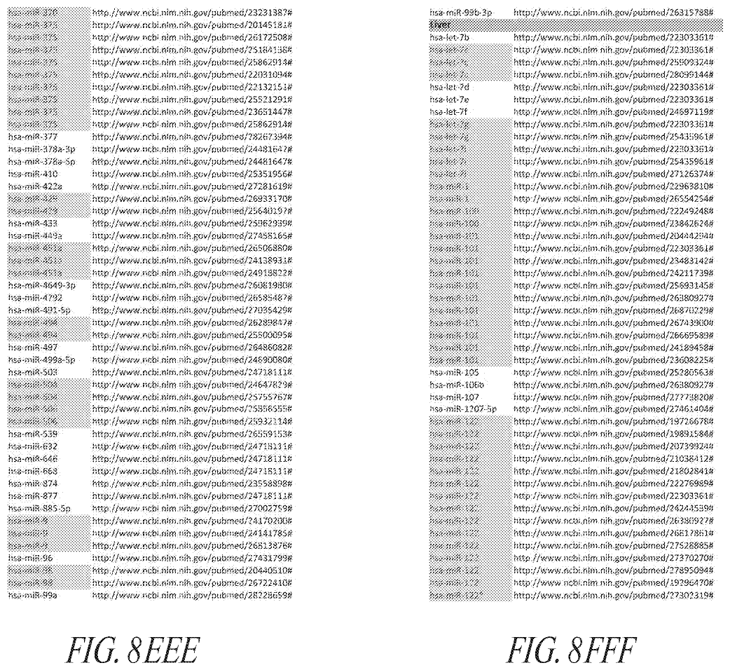

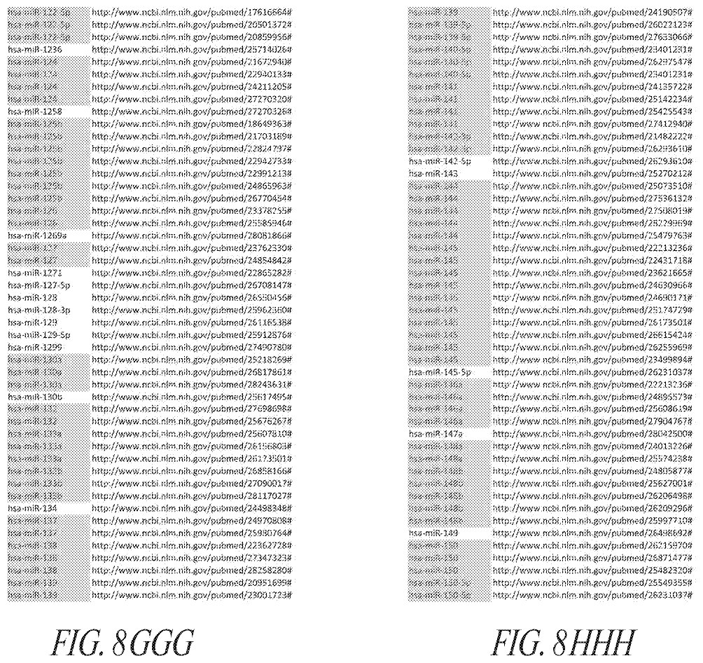

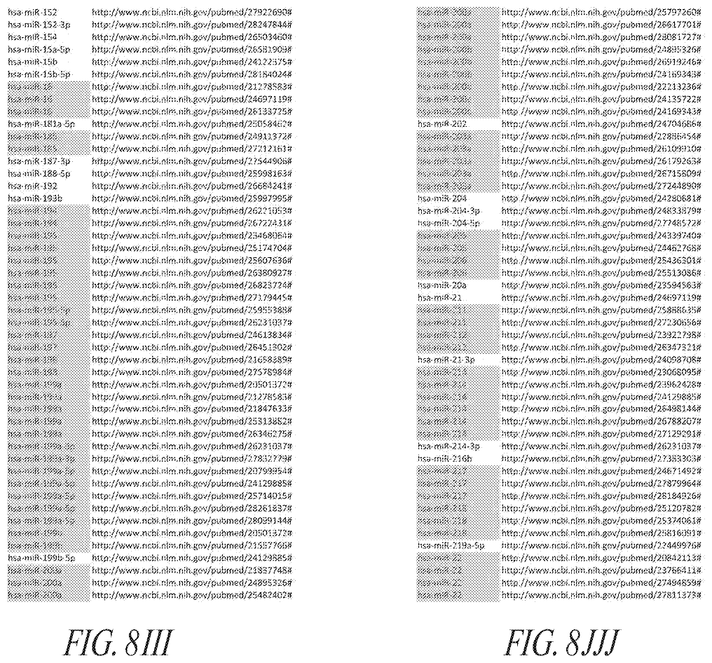

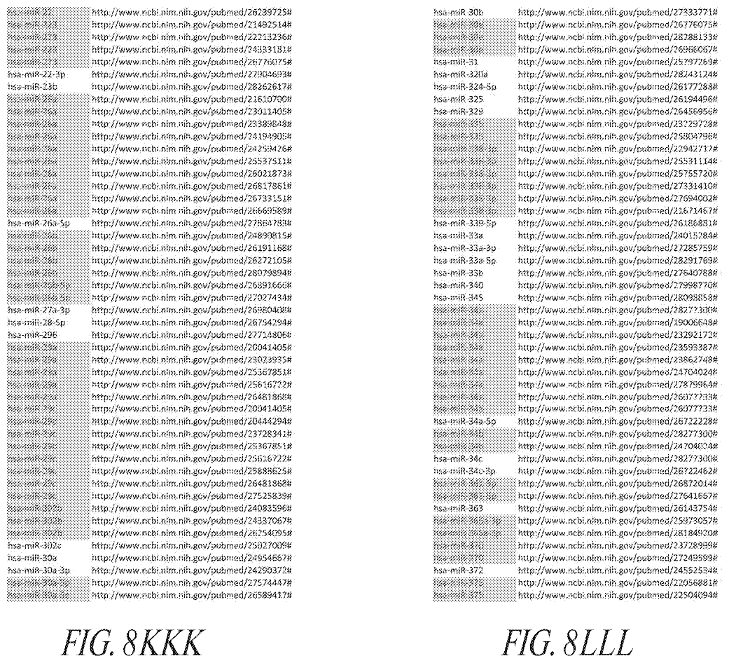

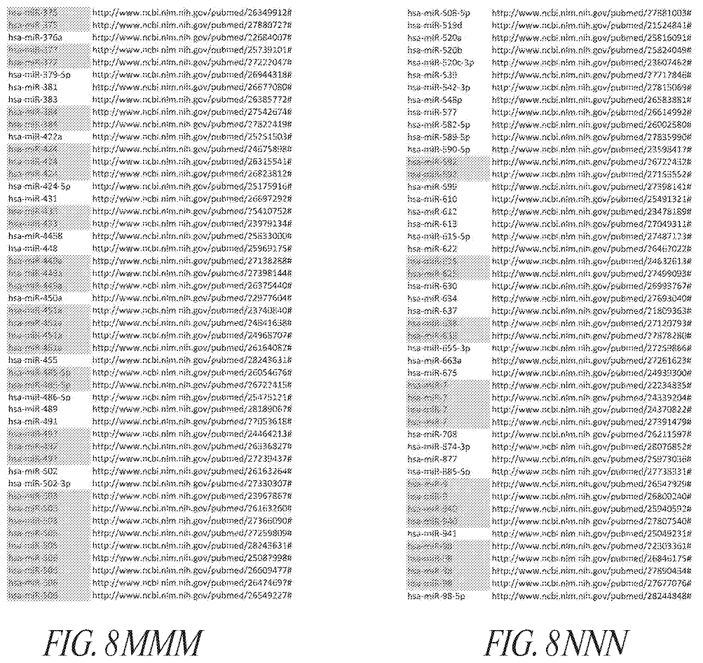

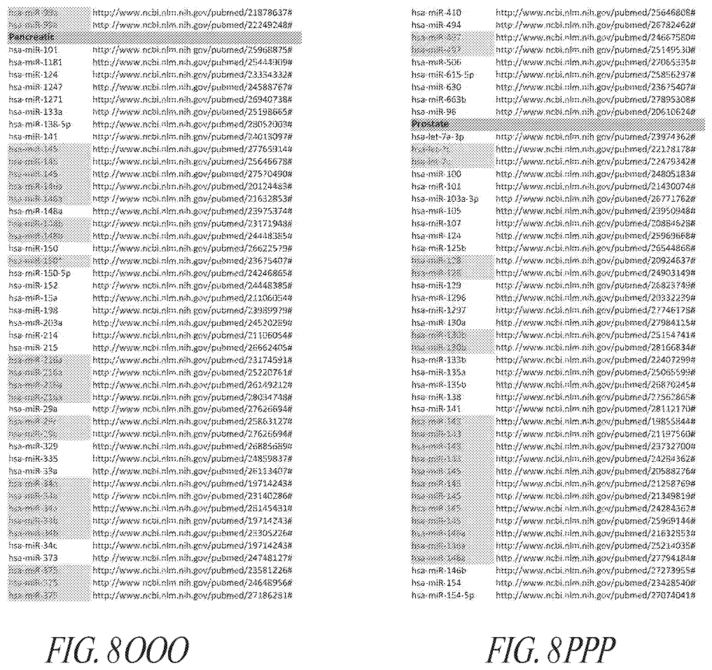

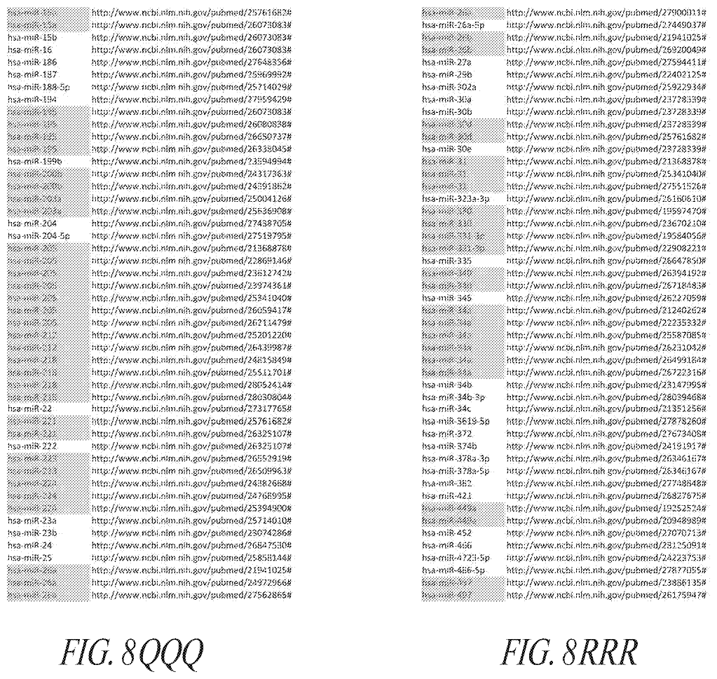

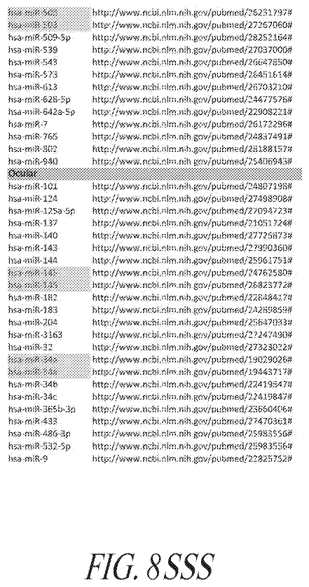

The term “microRNA” or “miRNA” as used herein refers to a family of short (typically 21-25 nucleotides), endogenous, single-stranded RNAs expressed in a wide range of organisms including both animals and plants. There are over 1000 unique miRNAs expressed in humans. miRNAs bind to specific target sequences found in messenger RNAs (mRNAs). Binding to complementary or partially complementary sequences (target sequences) in mRNA molecules results in down-regulation of gene expression by cleavage of the mRNA, increased degradation from shortening of its polyA tail, and direct translational repression. A selected list of microRNAs in tumors (along with associated references) is provided in FIGS. 8A-8Z, 8AA-8ZZ, and 8AAA-8SSS, which list and associated references are incorporated by reference in their entirety.

“MicroRNA target sequence(s),” “miRNA target sequence(s)” and “miRNA binding sequence(s)” refer to sequences which are complementary to, or bind to (i.e., they need not be 100% complementary) miRNA sequences such as those disclosed in FIG. 8.

The term “oncolytic Enterovirus” refers to an Enterovirus that is capable of replicating in and killing tumor cells. Briefly, Enterovirus is a genus of single stranded positive-sense RNA viruses which are most commonly associated with mammalian diseases that are transmitted through a fecal-oral route. Common examples of Enterovirus include poliovirus, coxsackievirus and echoviruses.

The term “oncolytic Coxsackievirus” or “CSV” refers generally to a Coxsackievirus capable of replicating in and killing tumor cells. Within certain embodiments the virus can be recombinantly (or ‘genetically’) engineered in order to more selectively target tumor cells and/or to reduce immune-mediated neutralization of the CSV in a human host. Coxsackievirus B3 (CVB3) is a small, nonenveloped virus that contains a positive RNA genome encoding a single open reading frame flanked by 5′ and 3′ untranslated regions (UTRs).

“Treat” or “treating” or “treatment,” as used herein, means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. The terms “treating” and “treatment” can also mean prolonging survival as compared to expected survival if not receiving treatment.

The term “cancer” refers to a disease state caused by uncontrolled or abnormal growth of cells in a subject. Representative forms of cancer include carcinomas, leukemias, lymphomas, myelomas, and sarcomas. Further examples include, but are not limited to cancer of the bile duct cancer, brain (e.g., glioblastoma), breast, cervix, colorectal, CNS (e.g., acoustic neuroma, astrocytoma, craniopharyngioma, ependymoma, glioblastoma, hemangioblastoma, medulloblastoma, meningioma, neuroblastoma, oligodendroglioma, pinealoma and retinoblastoma), endometrial lining, hematopoietic cells (e.g., leukemias and lymphomas), kidney, larynx, lung, liver, oral cavity, ovaries, pancreas, prostate, skin (e.g., melanoma and squamous cell carcinoma) and thyroid. Cancers can comprise solid tumors (e.g., sarcomas such as fibrosarcoma, myxosarcoma, liposarcoma, chondrosarcoma and osteogenic sarcoma), be diffuse (e.g., leukemias), or some combination of these (e.g., a metastatic cancer having both solid tumors and disseminated or diffuse cancer cells). Cancers can also be resistant to conventional treatment (e.g. conventional chemotherapy and/or radiation therapy).

Benign tumors and other conditions of unwanted cell proliferation may also be treated.

In order to further an understanding of the various embodiments herein, the following sections are provided which describe various embodiments: A. Oncolytic Enteroviruses; B. MicroRNAs; C. Therapeutic Compositions, and D. Administration.

### A. Oncolytic Enteroviruses

As noted above, Enteroviruses are a genus of single stranded positive-sense RNA viruses which are most commonly associated with mammalian diseases that are transmitted through a fecal-oral route. Common examples of Enteroviruses include polioviruses, coxsackieviruses and echoviruses.

Coxsackievirus is a virus that belongs to the Picornaviridae, which is a family of nonenveloped, linear, positive-sense single-stranded RNA viruses. More specifically, Coxsackievirus belongs to the genus Enterovirus, which also includes poliovirus and echovirus. Enteroviruses are among the most common and important human pathogens, and ordinarily its members are transmitted by the fecal-oral route. Coxsackieviruses are among the leading causes of aseptic meningitis (the other usual suspects being echovirus and mumps virus). Coxsackieviruses share many characteristics with poliovirus. With control of poliovirus infections in much of the world, more attention has been focused on understanding the nonpolio enteroviruses such as coxsackievirus. (Sean P, Semler BL. Coxsackievirus B RNA replication: lessons from poliovirus. Curr. Top. Microbiol. Immunol. 2008; 323:89-121).

Coxsackievirus B3 (CVB3) contains a positive-sense RNA genome encoding a single open reading frame flanked by 5′ and 3′ untranslated regions (UTRs). CVB3 has a short lifecycle, which typically culminates in rapid cell death and release of progeny virus. Subsequent to virus attachment to receptors, viral RNA is released into the cell where it acts as a template for the translation of the virus polyprotein and replication of the virus genome.

### B. MicroRNAs (miRNAs)

As noted above, the present invention provides miRNA-based approaches to modify the Enterovirus genome (e.g., a Poliovirus, Coxsackievirus or Echovirus genome) in order to reduce off-target toxicity while enhancing the stability of inserted miRNA target sites. miRNAs are a class of endogenous small non-coding RNAs that are evolutionarily conserved and act as key regulators in a wide range of fundamental cellular functions by binding to the UTR of the targeted mRNAs. Subsequently, they promote either mRNA degradation or suppression of gene expression. miRNAs can also play a key role in tumorigenesis. miRNAs are commonly observed to be downregulated in different cancer tissues. This unique feature can be exploited to develop miRNA-sensitive, tumor-targeted oncolytic viruses that spare specific normal tissues such as the heart and pancreas that are associated with toxicity in the context of certain wild-type Enteroviruses such as CVB3. As an example, miRNA-1 (miR-1), miRNA-133 (miR-133), miRNA-216 (miR-216), and miRNA-375 (miR-375) are tumor-suppressive miRNAs that are significantly downregulated in many cancer tissues, including small cell lung cancer (SCLC). Conversely, miR-1/miR-133 and miR-216/miR-375 are highly expressed in the heart and pancreas, respectively.

Individual miRNAs and groups of miRNAs may be expressed exclusively or preferentially in certain tissue types. Exemplary miRNAs include miR-1, miR-7, miR-30c, miR-124, miR-124*, miR-127, miR-128, miR-129, miR-129*, miR-133, miR-135b, miR-136, miR-136*, miR-137, miR-139-5p, miR-143, miR-154, miR-184, miR-188, miR-204, miR-208, miR-216, miR-217, miR-299, miR-300-3p, miR-300-5p, miR-323, miR-329, miR-337, miR-335, miR-341, miR-369-3p, miR-369-5p, miR-375, miR-376a, miR-376a*, miR-376b-3p, miR-376b-5p, miR-376c, miR-377, miR-379, miR-379*, miR-382, miR-382*, miR-409-5p, miR-410, miR-411, miR-431, miR-433, miR-434, miR-451, miR-466b, miR-485, miR-495, miR-499, miR-539, miR-541, miR-543*, miR-551b, miR-758, and miR-873. By convention, the strand that is more frequently found to be the final product is referred to as miRNA and the rarer partner as miRNA*.

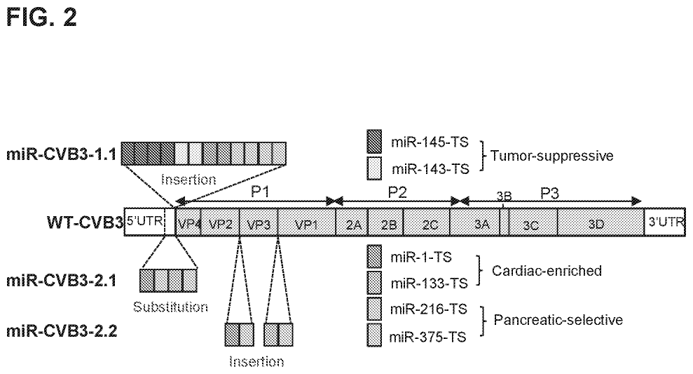

Within certain embodiments of the invention miRNA target sequences can be inserted into the UTR region (e.g., via substitution) and/or in-frame into the coding region of the Coxsackievirus B3 genome. Within other embodiments, the miRNA target sequences are inserted in-frame between the coding regions for two or more individual genes within the P1 region of the Coxsackievirus genome. Within certain embodiments at least one, two, three, four, five, or six miRNA target sequences can be inserted in tandem. Within further embodiments there may be at least 10 target sequences inserted in tandem. Within other embodiments there are less than 10, 15, 20, or 25 target sequences. Within preferred embodiments the miRNA target sequences are genetically stable (see, e.g., Schulze AJ. Insert Stability and In Vivo Testing of MicroRNA-Detargeted Oncolytic Picornaviruses. Methods Mol Biol. 2020;2058:77-94. doi: 10.1007/978-1-4939-9794-7_5. PMID: 31486032, which is incorporated by reference in its entirety). An optimal number of target sequences can be determined by assaying expression levels of CVB3. A low to nonexistent level of CVB3 in normal cells is desired. An optimal location for insertion at the UTR region and/or, an in-frame insertion at coding region target sequences into the CVB3 genome can be determined by passaging the engineered virus multiple times and verifying the presence of the inserted miRNA target sequences. A high level of stability for the inserted miRNA target sequences over multiple rounds of serial passage is desired. Genome stability may be tested over about 5, about 10, about 15, about 20, about 25, about 30, about 35, about 40, about 45, about 50, or more than about 50 rounds of serial passage. Persistence of the inserted miRNA target sequences across 20 or more rounds of serial passage is desired. The multiple miRNA target sequences may all bind the same miRNA or may bind different miRNAs. The target sequences may be in clusters (e.g., FIG. 2) in which for example, there are at least two target sequences in tandem that bind a first miRNA followed by at least two target sequences in tandem that bind a second miRNA and, optionally, followed by at least two target sequences that bind a third miRNA and at least two target sequences that bind a fourth miRNA. Alternatively, the multiple miRNA target sequences that bind different miRNAs may be in no particular order. As well, there may be only a single copy of each miRNA target sequence. In some embodiments, there are 2-10 different miRNA targets. In other embodiments, there are 2-10 copies of each target sequence. In other embodiments, there are 2-10 different miRNA targets and 2-10 copies of each of these target sequences in clusters. In further embodiments, there are 2-4 different miRNA targets and only a single copy of each miRNA target sequence. The miRNA target sequences may be inserted in any orientation or combination of orientations. See FIG. 2 for three exemplary constructs.

Within certain embodiments of the invention, two or more different miRNAs which are highly expressed in the same organ or tissue are targeted in order to provide redundancy in case the miRNA target sequences are rendered non-functional over multiple passages during the course of treatment.

The multiple miRNA target sequences may be adjacent without intervening nucleotides or have from 1 to about 25, or from 1 to about 20, or from 1 to about 15, or from 1 to about 10, or from 1 to about 5, or from 3 to about 10, or from 5 to about 10 intervening nucleotides. Intervening nucleotides may be chosen to have a similar G+C content as the 5′UTR and preferably do not contain a polyadenylation signal sequence.

The multiple miRNA target sequences may be inserted UTR bby substitution without changing the genome length for maximal capacity for the incorporation of exogenous gene. More preferably, the multiple miRNA target sequence may be inserted into the ribosomal scanning region between the IRES located in the 5′ UTR and the start codon of the viral genome, where the length matters more than the content. Also, the multiple miRNA target sequences may be inserted in-frame directly into the coding region(s) for one or more individual genes within the Coxsackievirus genome, preferably at the 5′-end of the gene coding region(s). More preferably, the multiple miRNA target sequences may be inserted in-frame between the coding regions for two or more individual genes within the Coxsackievirus genome. For example, the multiple miRNA target sequences may be inserted in-frame between the genes encoding proteins VP4 and VP2, between the genes encoding proteins VP2 and VP3, between the genes encoding proteins VP3 and VP1, between the genes encoding proteins VP1 and 2A, between the genes encoding proteins 2A and 2B, between the genes encoding proteins 2B and 2C, between the genes encoding proteins 2C and 3A, between the genes encoding proteins 3A and 3B, between the genes encoding proteins 3B and 3C, and/or between the genes encoding proteins 3C and 3D).

### C. Therapeutic Compositions

Therapeutic compositions are provided that may be used to prevent, treat, or ameliorate the effects of a disease, such as, for example, cancer. More particularly, therapeutic compositions are provided comprising at least one oncolytic virus as described herein.

In certain embodiments, the compositions will further comprise a pharmaceutically acceptable carrier. The phrase “pharmaceutically acceptable carrier” is meant to encompass any carrier, diluent or excipient that does not interfere with the effectiveness of the biological activity of the oncolytic virus and that is not toxic to the subject to whom it is administered (see generally Remington: The Science and Practice of Pharmacy, Lippincott Williams & Wilkins; 21st ed. (May 1, 2005 and in The United States Pharmacopoeia: The National Formulary (USP 40-NF 35 and Supplements).

In the case of an oncolytic virus as described herein, non-limiting examples of suitable pharmaceutical carriers include phosphate buffered saline solutions, water, emulsions (such as oil/water emulsions), various types of wetting agents, sterile solutions, and others. Additional pharmaceutically acceptable carriers include gels, bioabsorbable matrix materials, implantation elements containing the oncolytic virus, or any other suitable vehicle, delivery or dispensing means or material(s). Such carriers can be formulated by conventional methods and can be administered to the subject at an effective dose. Additional pharmaceutically acceptable excipients include, but are not limited to, water, saline, polyethylene glycol, hyaluronic acid and ethanol. Pharmaceutically acceptable salts can also be included therein, e.g., mineral acid salts (such as hydrochlorides, hydrobromides, phosphates, sulfates, and the like) and the salts of organic acids (such as acetates, propionates, malonates, benzoates, and the like). Such pharmaceutically acceptable (pharmaceutical-grade) carriers, diluents and excipients that may be used to deliver the oncolytic virus to a cancer cell will preferably not induce an immune response in the individual (subject) receiving the composition (and will preferably be administered without undue toxicity).

The compositions provided herein can be provided at a variety of concentrations. For example, dosages of oncolytic virus can be provided which ranges from about 10⁶ to about 10¹¹ pfu. Within further embodiments, the dosage can range from about 10⁶ to about 10¹⁰ pfu/ml, with up to 4 mls being injected into a patient with large lesions (e.g., >5 cm) and smaller amounts (e.g., up to 0.1 mls) in patients with small lesions (e.g., <0.5 cm) every 2-3 weeks, of treatment.

Within certain embodiments of the invention, lower dosages than standard may be utilized. Hence, within certain embodiments less than about 10⁶ pfu/ml (with up to 4 mls being injected into a patient every 2-3 weeks) can be administered to a patient.

The compositions may be stored at a temperature conducive to stable shelf-life and includes room temperature (about 20° C.), 4° C., −20° C., −80° C., and in liquid N2. Because compositions intended for use in vivo generally don't have preservatives, storage will generally be at colder temperatures. Compositions may be stored dry (e.g., lyophilized) or in liquid form.

### D. Administration

In addition to the compositions described herein, various methods of using such compositions to treat or ameliorate disease (e.g., cancer) are provided, comprising the step of administering an effective dose or amount of a modified Coxsackievirus as described herein to a subject.

The terms “effective dose” and “effective amount” refers to amounts of the oncolytic virus that is sufficient to effect treatment of a targeted cancer, e.g., amounts that are effective to reduce a targeted tumor size or load, or otherwise hinder the growth rate of targeted tumor cells. More particularly, such terms refer to amounts of oncolytic virus that is effective, at the necessary dosages and periods of treatment, to achieve a desired result. For example, in the context of treating a cancer, an effective amount of the compositions described herein is an amount that induces remission, reduces tumor burden, and/or prevents tumor spread or growth of the cancer. Effective amounts may vary according to factors such as the subject's disease state, age, gender, and weight, as well as the pharmaceutical formulation, the route of administration, and the like, but can nevertheless be routinely determined by one skilled in the art.

The therapeutic compositions are administered to a subject diagnosed with cancer or is suspected of having a cancer. Subjects may be human or non-human animals.

The OV (e.g., Coxsackievirus) as described herein may be given by a route that is e.g. intravenous, intratumoral, or intraperitoneal. Within certain embodiments the oncolytic virus may be delivered by a cannula, by a catheter, or by direct injection. The site of administration may be directly into the tumor or at a site distant from the tumor. The route of administration will often depend on the type of cancer being targeted. The OV (e.g., Coxsackievirus) as described herein are particularly suitable for intravenous (IV) administration.

The optimal or appropriate dosage regimen of the oncolytic virus is readily determinable by those skilled in the art, by the attending physician based on patient data, patient observations, and various clinical factors, including for example a subject's size, body surface area, age, gender, and the particular oncolytic virus being administered, the time and route of administration, the type of cancer being treated, the general health of the patient, and other drug therapies to which the patient is being subjected. According to certain embodiments, treatment of a subject using the oncolytic virus described herein may be combined with additional types of therapy, such as radiotherapy or chemotherapy using, e.g., a chemotherapeutic agent such as etoposide, ifosfamide, adriamycin, vincristine, doxycycline, and others.

OV (e.g., Coxsackievirus) may be formulated as medicaments and pharmaceutical compositions for clinical use and may be combined with a pharmaceutically acceptable carrier, diluent, excipient, or adjuvant. The formulation will depend, at least in part, on the route of administration. Suitable formulations may comprise the virus and inhibitor in a sterile medium. The formulations can be fluid, gel, paste or solid forms. Formulations may be provided to a subject or medical professional.

A therapeutically effective amount is preferably administered. This is an amount that is sufficient to show benefit to the subject. The actual amount administered and time-course of administration will depend at least in part on the nature of the cancer, the condition of the subject, site of delivery, and other factors.

Within yet other embodiments of the invention the oncolytic virus can be administered by a variety of methods, e.g., intratumorally, intraperitoneally, intravenously, or after surgical resection of a tumor.

The following are additional exemplary embodiments of the present disclosure:
1. A replicating oncolytic virus vector comprising a modified Enterovirus genome, wherein the modified Enterovirus genome comprises one or more copies of one or more miRNA target sequences inserted into the UTR region (e.g., via substitution) and/or in-frame into the coding region of the Enterovirus genome. Within a related embodiment, replicating oncolytic virus vectors are provided comprising a modified Enterovirus genome, wherein the modified Enterovirus genome comprises a plurality of one or more miRNA target sequences inserted in-frame into the coding region of the Enterovirus genome. Within various embodiments the Enterovirus may be a Poliovirus, a Coxsackievirus, or an Echovirus. Within further embodiments, the replicating oncolytic virus vectors exhibit reduced toxicity after multiple passages when compared to a miR-regulated coxsackievirus with the miRNA inserted into the 5′-UTR and/or 3′-UTR region(s). This is due to increased stability of the miRNA insert since it is inserted the UTR region via substitution and/or into the coding region via in-frame insertion. The insertion into UTR is done via substitution without disrupting the evolutionarily optimal length of the UTR/viral genome length, reducing the possibility of loss of the miRNA target sequence during viral replication. The insertion into the coding region is done in-frame, so partial revertants have a high probability of being non-functional due to frameshift. The insertion into coding region is more stable than the insertion into UTR via substitution. However, this is still not 100% reliable because complete deletion of the miRNA insert or partial deletion in multiples of 3 nucleotides would not result in a frameshift. However this strategy results in much greater genome stability and lower toxicity than the previous approach of inserting the miRNA into a noncoding region. Notably, this reduced toxicity only manifests after multiple serial passages due to enhanced stability of the inserted miRNA target sequences. Within yet other related embodiments, oncolytic viral vectors are provided wherein a portion of the native 5′-UTR and/or 3′-UTR is replaced with miRNA target sequences. The portion of the native 5′-UTR and/or 3′-UTR that is deleted to be replaced with miRNA target sequences is of approximately the same length as said miRNA target sequences to avoid substantially changing the total length of the 5′-UTR and/or 3′-UTR after insertion of miRNA target sequences. Within some embodiments, the total length of the 5′-UTR and/or 3′-UTR after insertion of miRNA target sequences is identical to the total length of the native 5′-UTR and/or 3′-UTR. Within other embodiments, the total length of the 5′-UTR and/or 3′-UTR after insertion of miRNA target sequences differs from the total length of the native 5′-UTR and/or 3′-UTR by less than 1%, by less than 5%, by less than 10%, by less than 15%, by less than 20%, or by less than 25%.
2. The replicating oncolytic virus vector of embodiment 1, wherein said Enterovirus is a Coxsackievirus.
3. The replicating oncolytic virus vector of embodiment 2, wherein the Coxsackievirus is Coxsackievirus A or B.
4. The replicating oncolytic virus vector of any one of embodiments 1, 2 or 3, wherein the coding region is the region between the genes encoding proteins VP4 and VP2, VP2 and VP3, VP3 and VP1, VP1 and 2A, 2A and 2B, 2B and 2C, 2C and 3A, 3A and 3B, 3B and 3C, and/or 3C and 3D. Within other embodiments, one or more miRNA target sequences may be inserted into one or more intergenic regions within the Enterovirus genome open reading frame encoding the Enterovirus genome polyprotein. Within further embodiments, one or more miRNA target sequences may be inserted between the genes encoding proteins VP2 and VP3 and/or one or more miRNA target sequences may be inserted between the genes encoding proteins VP3 and VP1 and/or one or more miRNA target sequences may be inserted between the genes encoding proteins 2A and 2B and/or one or more miRNA target sequences may be inserted between the genes encoding proteins 2C and 3A.
5. The replicating oncolytic virus vector of any one of embodiments 1, 2, 3 or 4, wherein the one or more copies of the one or more miRNA target sequences comprises one or more copies of two or more different miRNA target sequences.
6. The replicating oncolytic virus vector of any one of embodiments 1, 2, 3, 4, or 5, wherein spacers of 1 to 50 base pairs (“bp”) in size are inserted between the one or more miRNA target sequences. Within various embodiments the spacers may be 1-10 bp in size, 10-20 bp in size, 20-30 bp in size, 30-40 bp in size, or 40-50 bp in size.
7. The replicating oncolytic virus vector of embodiment 1 wherein the one or more copies of the one or more miRNA target sequences target cardiac or pancreatic specific miRNAs. Representative examples of cardiac specific miRNAs include miR-1, miR-133a/b, miR-208a/b and miR-499. Representative examples of pancreatic specific miRNAs include miR-7, miR-204, miR-216, miR-217, and miR-375.
8. The replicating oncolytic virus vector of any one of embodiments 1, 2, 3, 4, 5, 6, or 7, wherein the one or more different miRNA target sequences target an miRNA selected from the group consisting of miR-1, miR-7, miR-30c, miR-124, miR-124*, miR-127, miR-128, miR-129, miR-129*, miR-133, miR-135b, miR-136, miR-136*, miR-137, miR-139-5p, miR-143, miR-154, miR-184, miR-188, miR-204, miR-208, miR-216, miR-217, miR-299, miR-300-3p, miR-300-5p, miR-323, miR-329, miR-337, miR-335, miR-341, miR-369-3p, miR-369-5p, miR-375, miR-376a, miR-376a*, miR-376b-3p, miR-376b-5p, miR-376c, miR-377, miR-379, miR-379*, miR-382, miR-382*, miR-409-5p, miR-410, miR-411, miR-431, miR-433, miR-434, miR-451, miR-466b, miR-485, miR-495, miR-499, miR-539, miR-541, miR-543*, miR-551b, miR-758, and miR-873. By convention, the strand that is more frequently found to be the final product is referred to as miRNA and the rarer partner as miRNA*. Within various embodiments, the replicating oncolytic virus may contain one or more copies of positive strand miRNAs and/or one or more copies of negative strand miRNAs.
9. The replicating oncolytic virus vector of any one of embodiments 1, 2, 3, 4, 5, 6, 7, or 8, wherein the two or more (or plurality) of different miRNA target sequences comprise target sequences for miR-1, miR-133, miR-216, and miR-375.
10. The replicating oncolytic virus vector of embodiment 9, comprising one, two, three, four, five, or six copies of the target sequence for miR-1, miR-133, miR-216, and miR-375.
11. The replicating oncolytic virus vector of any one of embodiments 1, 2, 3, 4, 5, 6, 7, 8, 9, or 10, wherein one or more copies of the one or more miRNA target sequences is in a forward orientation and one or more copies of the one or more miRNA target sequences is in a reverse orientation.
12. The replicating oncolytic virus vector of any one of embodiments 1, 2, 3, 4, 5, 6, 7, 8, 9, 10, or 11, wherein the modified Enterovirus genome comprises at least one nucleic acid encoding a non-viral protein selected from the group consisting of immunostimulatory factors, antibodies (including for example bispecific antibodies), and checkpoint blocking peptides (also referred to as “checkpoint inhibitors” or “checkpoint modulators”), wherein the at least one nucleic acid is operably linked to a suitable tumor-specific regulatory region. Within various embodiments of the invention the bispecific antibody comprises a first antigen-binding domain which recognizes a tumor antigen, as well as a second antigen-binding domain which recognizes a cell surface molecule on an effector cell. Within other embodiments of the invention the checkpoint modulator is a peptide ligand, soluble domain of natural receptor, RNAi, antisense molecule or antibody. Within further embodiments of the invention the immune modulator at least partially antagonizes the activity of an inhibitory immune checkpoint(s), such as, for example, PD-1, PD-L1, PD-L2, LAG 3, Tim3, BTLA and/or CTLA4.
13. The replicating oncolytic virus vector of embodiment 12, wherein the non-viral protein is selected from the group consisting of IL12, IL15, IL15 receptor alpha subunit, OX40L, CD73, and a checkpoint inhibitor. Within further embodiments of the invention the above noted replicating oncolytic virus described in any of embodiments 1, 2, 3, 4, 5, 6, 7, 8, 9, 10, 11, or 12 retains its ability to infect and lyse tumor cells, but has reduced toxicity in vitro and/or in vivo as compared to an unmodified wild-type virus of the same strain and also has reduced toxicity in vitro and/or in vivo as compared to a virus of the same strain that has been modified by inserting miRNA target sequences into the 5′-untranslated region or into the 3′-untranslated region. Within certain embodiments, the reduced toxicity is in cardiomyocytes, pancreatic cells, lung cells, and/or stem cells. Within further embodiments the reduced toxicity is only evident after multiple serial passages, (e.g., at least 5, 10, 20, 30, 40, 50, 60, 70, 80, or 90 passages).
14. A method for lysing tumor cells, comprising providing an effective amount of a replicating oncolytic virus vector of any of embodiments 1, 2, 3, 4, 5, 6, 7, 8, 9, 10, 11, 12, or 13 to tumor cells. Tumor cells can be found, for example, in vivo within the cancers described herein,
15. The method of embodiment 14, wherein the tumor cells comprise lung cancer cells.
16. The method of embodiment 14, wherein the tumor cells comprise pancreatic cancer cells.
17. A therapeutic composition comprising at least one replicating oncolytic virus vector of any of the above embodiments 1, 2, 3, 4, 5, 6, 7, 8, 9, 10, 11, 12, or, 13, and a pharmaceutically acceptable carrier.
18. A method for treating cancer in a subject suffering therefrom, comprising the step of administering a composition comprising a therapeutically effective amount of the composition of embodiment 17. Representative examples of cancers include those that are described herein. Particularly preferred cancers include lung cancers, pancreatic cancer, liver cancer and breast cancer.
19. The method of embodiment 18, wherein the cancer is non-small-cell lung cancer (NSCLC) associated with KRAS mutations, small-cell lung cancer (SCLC) commonly linked to TP53 and Rb mutations, or pancreatic cancer.
20. The method of embodiment 18, wherein the administration is intravenous (IV) administration, intraperitoneal (IP) administration, or intratumoral (IT) administration.

The following Examples are offered by way of illustration and not by way of limitation.

### EXAMPLES

### Example 1

Expression Profiling of miR-1, miR-133, miR-216, and miR-375 in Mouse Tissues and Tumor Cells

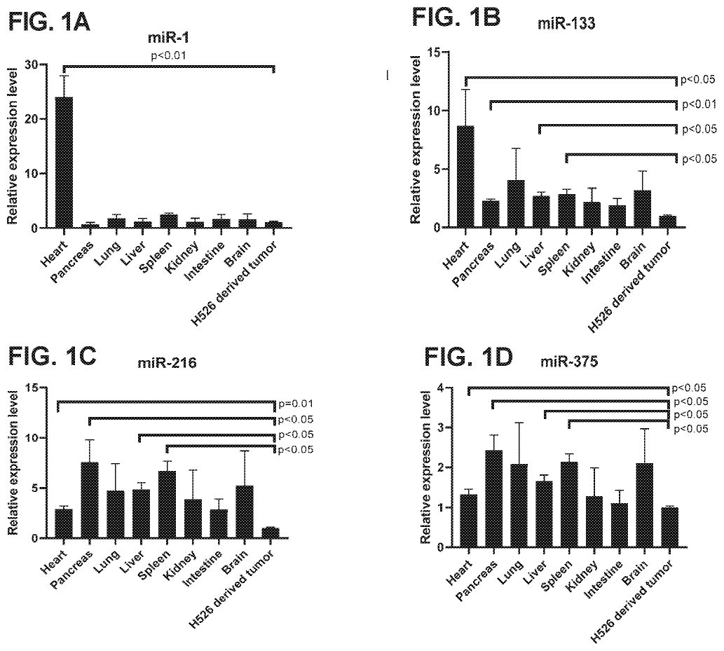

This example describes an experiment to investigate the expression of miR-1,miR-133, miR-216, and miR-375 in various mouse tissues and in H526 small cell lung cancer cells. To verify organ-selective expression, the relative levels of miR-1 (FIG. 1A), miR-133 (FIG. 1B), miR-216 (FIG. 1C), and miR-375 (FIG. 1D) in various SCID mouse tissues and H526-derived xenograft tumors was measured by RT-qPCR and presented as mean ±SD (n=3). The results indicate that miR-1 and miR-133 are significantly abundant in the heart as compared to other organs and tumor tissues and that expression levels of miR-216 and miR-375 are significantly higher in the pancreas than in the tumor tissues.

### Example 2

Construction of Three Different miRNA-Modified CVB3

To generate a recombinant CVB3 vector with decreased viral toxicity in normal tissues, a miRNA-engineered CVB3 named miR-CVB3-2.1 was constructed by replacing the last 88 nucleotides of the CVB3 5′UTR (i.e., ribosomal scanning region after domain VI and before start codon) with target sequences (TS) of miR-1, miR-133, miR-216, and miR-275 (one copy of each). The miRNA-engineered CVB3 named miR-CVB3-2.2 was developed by inserting TS of the above miRs into the P1 region (i.e., 1×miR-1-TS and 1×miR-216-TS inserted between the genes encoding VP2 and VP3, and 1×miR-133-TS and 1×miR-375-TS inserted between the genes encoding VP3 and VP1). The miR-CVB3-1.1 was generated by inserting 4×miR-145-TS, 2×miR-143-TS, 2×miR-1-TS, and 4×miR-216-TS into the 3′ terminus of CVB3 5′UTR (FIG. 2).

Specifically, a linearized CVB3 plasmid (Kandolf strain) was first amplified by a primer pair around the insertion site. The miR-CVB3 was then assembled with an oligo pair including the miRNA TS and necessary overlapping sequence using the NEBuilder HiFi DNA Assembly Master Mix kit (E2621, New England Biolabs) following the manufacturer's protocol. To preserve the length of 5′UTR, miR-CVB3-2.1 was constructed by substituting 88-nucleotide between IRES and start codon at the 5′UTR with TS of miR-1, miR-133, miR-216, miR-375 (one copy of each without spacer in between) first using a primer pair (forward: 5′-ATA CAG CAA AAT GGG AGC TCA-3′, reverse: 5′-CAC CGG ATG GCC AAT CCA AT-3′) and then an oligo pair (fragment 1: 5′-ATT GGA TTG GCC ATC CGG TGA TAC ATA CTT TAC ATT CCA TCA CAG TTG CCA GCT GAG ATT ATA GCT GGT TG-3′, fragment 2: 5′-TGA GCT CCC ATT TTG CTG TAT TTT GTT CGT TCG GCT CGC GTG ATT TGG TCC CCT TCA ACC AGC TAT AAT CTC AGC-3′). miR-CVB3-2.2 was generated by first inserting TS of miR-1 and miR-216 (one copy of each) between VP2 and VP3 using a primer pair (forward: 5′-GGC TTA CCA ACC ATG AAT AC-3′, reverse: 5′-CTG GTG CCC TGC TAA ACG TAA C-3′) and an oligo pair (fragment 1: 5′-GTT ACG TTT AGC AGG GCA CCA GGG CCT TAA AGA CAT ACA TAC TTC TTT ACA TTC CAA TAG TCA CAG TTG CCA GCT GAG ATT AGC AGA GTT TCA AGG CTT ACC AAC CAT GAA TAC-3′), followed by further insertion of TS of miR-133 and miR-375 (one copy of each) between VP3 and VP1 with a primer pair (forward: 5′-GGC CCA GTG GAA GAC GCG ATA-3′, reverse: 5′-CTG GAA AAA GTT TTG CTG CG-3′) and an oligo pair (fragment: 5′-CGC AGC AAA ACT TTT TCC AGG GTC CAC CAG TAC TTA GCT GGT TGA AGG GGA CCA AAA CTA GTC ACG CGA GCC GAA CGA ACA AAG CAC TAT TCC AGG GCC CAG TGG AAG ACG CGA TA-3′).

To produce live viral stock, the viral genome was synthesized using HiScribe™ T7 Quick High Yield RNA Synthesis Kit (#E2050S, New England Biolabs). Subsequently, viral RNA was transfected into Hela cells and the supernatant was collected at ˜72 hours post-transfection when cytopathic effects were most prominent. The virus-containing supernatant was further propagated in Hela cells until viral titers reached desirable levels for storage.

### Example 3

Assessment of Genomic Stability of Newly Generated miR-CVB3s In Vitro

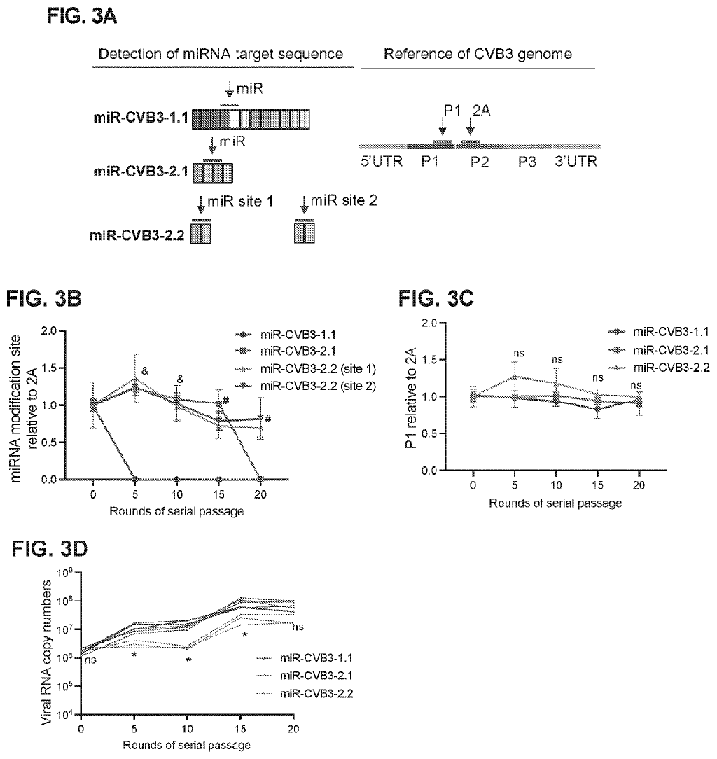

Due to the increased selective pressure, HL-1 mouse cardiomyocytes, which express miR-1 and miR-133, were selected to evaluate the stability of miR-CVB3s. To test the genomic stability, the newly established miR-CVB3s were serially passaged in HL-1 cardiomyocytes (i.e., supernatant from infected cells was collected and transferred to new cells) for 20 rounds (n=3 wells for each round). The cell supernatants were collected every 5 passages at 24 hr post-infection for viral genome quantification by RT-qPCR with primer pairs as follows: miR-CVB3-1.1(forward: 5′-CCC TTT GTT GGG TTT ATA CCA CTT-3′, reverse: 5′-CCA GGA ATC CCT TTG ACG TCC A-3′), miR-CVB3-2.1 (forward: 5′-CCA TAT AGC TAT TGG ATT GGC CAT-3′, reverse: 5′-CGT TGA TAC TTG AGC TCC CAT-3′), miR-CVB3-2.2 site 1 (forward: 5′-GCC GAG TAC AAT GGG TTA CG-3′, reverse: 5′-CTG GCA ACT GTG ACT ATT GGA A-3′), miR-CVB3-2.2 site 2 (forward: 5′-GTT GAA GGG GAC CAA AAC TAG TC-3′, reverse: 5′-CTC CCT ATA GCG GCT GTT ATC G-3′), 2A of CVB3 (forward: 5′-GCT TTG CAG ACA TCC GTG ATC-3′, reverse: 5′-CAA GCT GTG TTC CAC ATA GTC CTT CA-3′), P1 of CVB3 (forward: 5′-GAA GGA CAC TCC TTT CAT TTC GC-3′, reverse: 5′-CTC CCT ATA GCG GCT GTT ATC G-3′). The relative location of RT-PCR primers targeting specific miR-TS or P1 region is depicted in FIG. 3A. Primers binding to P1 region or 2A within P2 region were used as references (internal controls). Two primer pairs (denoted as “site 1” and “site 2”) were designed to assess the integrity of miR-CVB3-2.2 by monitoring the existence of two separate miR-TS inserts.

FIG. 3B shows quantification of miRNA modification sites of different miR-CVB3. The value of “1.0” indicates intact primer binding sites on miR-TS, whereas “0” denotes reversion mutation or loss of miR-TS. The number between “1.0” and “0” suggests reduced viral genome stability. As shown in FIG. 3B, after 5-round passaging, the value of miR-CVB3-1.1 reached “0”, indicating complete loss or instability of miRNA modification sites. In contrast, miR-CVB3-2.1 was stable until passage 15 and miR-CVB3-2.2 until at least passage 20, suggesting a great improvement in the stability of miR-CVB3 that incorporates miRNA target sequences within the coding region of the CVB3 genome that encodes the CVB3 genome polyprotein. As the miR-TS in miR-CVB3-2.2 were inserted into two sites within the P1 region, the stability of the two inserts was examined separately using two primer pairs. As comparison, relative quantification of P1 to 2A as an internal control remained around 1.0 across the 20-round passaging (FIG. 3C). The absolute quantification of viral genome copies presented in FIG. 3D revealed that viruses replicate normally across the 20-round passaging, excluding the possibility that reduction in copy number of miRNA modification sites on miR-CVB3-1.1 and miR-CVB3-2.1 was a result of halted viral replication.

### Example 4

Evaluation of Lytic Ability of New miR-CVB3s In Vitro

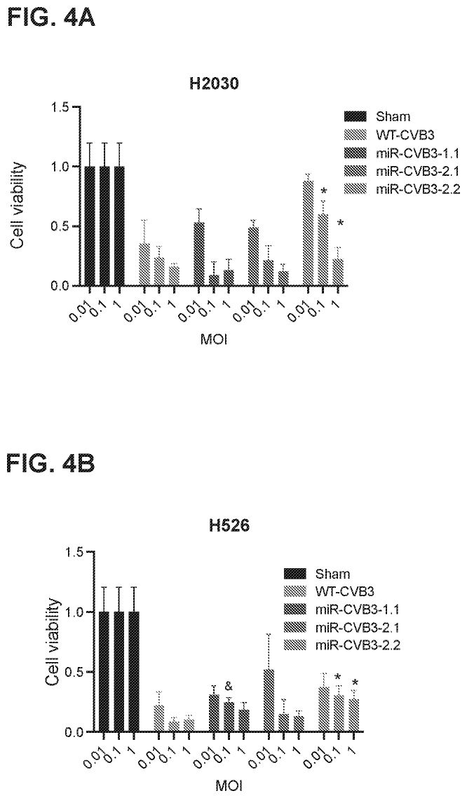

To determine whether the miRNA modification impairs the lytic ability of miR-CVB3 against tumor cells, human KRAS^{mut} lung adenocarcinoma H2030 (FIG. 4A) and TP53^{mut}/RB1^{mut} SCLC H526 cells (FIG. 4B) were sham-infected or incubated with WT-CVB3, miR-CVB3-1.1, miR-CVB3-2.1, or miR-CVB3-2.2 at a multiplicity of infection (MOI) of 0.01, 0.1 and 1 for 72 hrs. Cell viability was assessed by alamarBlue assay (mean±SD, n=3), demonstrating the effectiveness of these new miR-CVB3s in lysing tumor cells, especially at the MOI of 1, albeit at a slightly reduced level relative to WT-CVB3. It can be speculated that artificial miRNA modification of CVB3 selectively boosts the innate immunity or antiviral ability against the miRNA-modified CVB3 in both normal cells and cancer cells, thereby accounting for the slightly reduced lytic ability of miR-CVB3 observed in cancer cells compared to WT-CVB3. Because the abundance of miR-1 and miR-133 in cardiomyocytes and the abundance of miR-216 and miR-375 in non-cancerous pancreatic cells is substantially higher than in cancer cells, it is predicted that cardiomyocytes and pancreatic cells should be less susceptible to damage by miR-CVB3.

### Example 5

Evaluation of Replicative Ability of New miR-CVB3s In Vitro

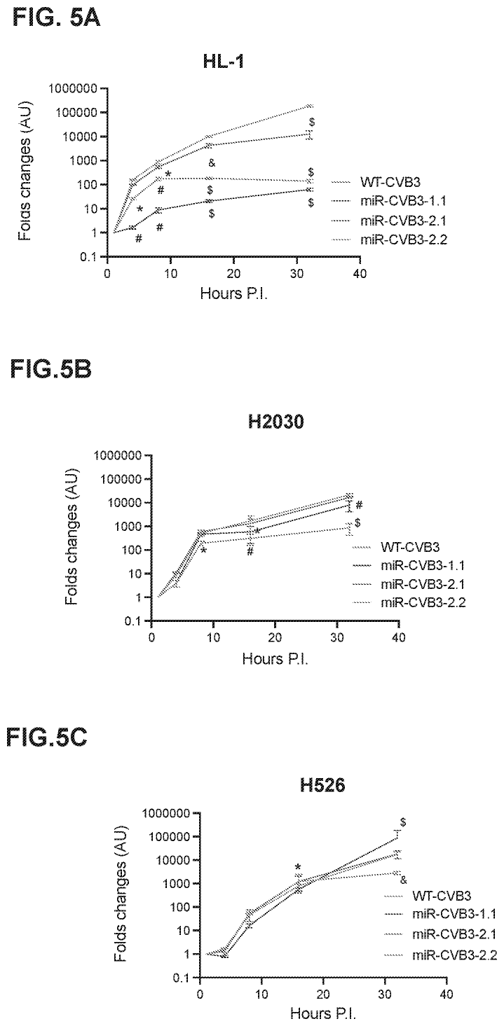

To further determine the replication kinetics of miR-CVB3s, RT-qPCR was performed to measure the growth curve of viral genomes over a 32-hour experimental period. HL-1 cardiomyocytes, H2030, and H526 cells were infected with WT- or different miR-CVB3s at an MOI of 0.01 for the indicated time periods (FIG. 5A, 5B, 5C). Cell lysates were harvested for RNA extraction and absolute quantification of viral genomes by RT-qPCR using 2A primer pairs (mean±SD, n=3). Unpaired Student's t-test was conducted. *, p<0.05; #, p<0.01; &, p<0.005; $, p<0.001 compared to WT-CVB3. PI, post-infection.

Viral RNA levels were substantially lower in HL-1 cells inoculated with various miR-CVB3s compared with WT-CVB3 (FIG. 5A). It was noted that, between the two new miR-CVB3s, replication of miR-CVB3-2.2 (similar observation was made for miR-CVB3-1.1) in HL-1 cells was significantly less efficient than that of miR-CVB3-2.1, even though they harbor the same miR-TS. Furthermore, RNA copy numbers of miR-CVB3-2.1 and miR-CVB3-2.2 were comparable or modestly reduced in H2030 cells (FIG. 5B) and H526 cells (FIG. 5C) as compared to WT-CVB3. Together, these results suggest that the second generation miR-CVB3s retain the oncolytic and replication activity in lung cancer cells whereas their ability to replicate in cardiomyocytes was drastically attenuated as compared to WT-CVB3.

### Example 6

Safety Assessment of New miR-CVB3s in Immunocompetent Mice

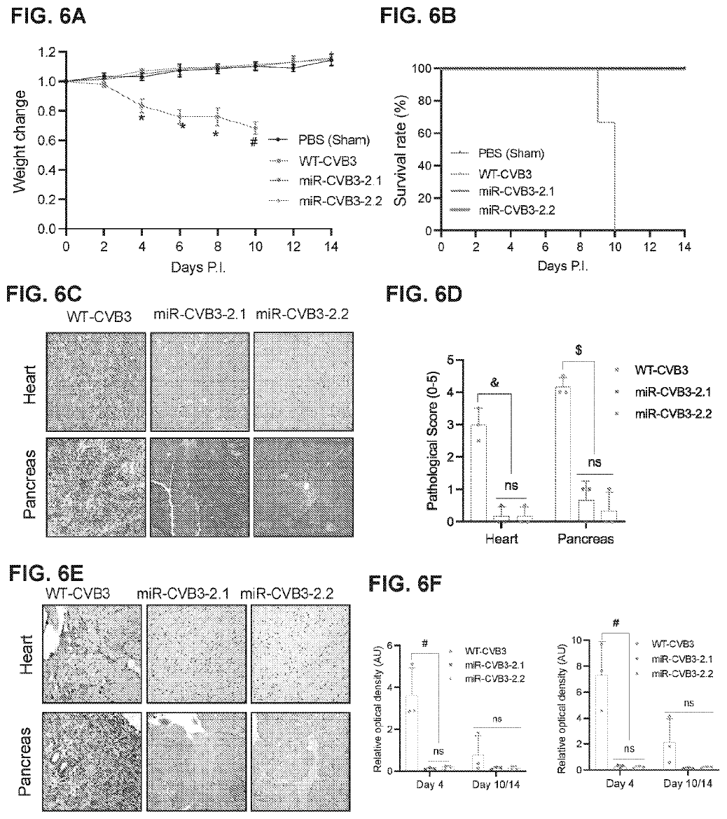

A/J mice, a mouse strain known to be susceptible to CVB3 infection, were inoculated intraperitoneally with PBS, WT- or miR-CVB3s once at a dose of 1×10⁶ plaque-forming units (PFU) for up to 14 days (experimental endpoint). Body weight was measured every other day (mean±SE, n=3 for each group) and normalized to that on day 0, which was arbitrarily set a value of 1.0 (FIG. 6A). Kaplan-Meier survival rate (n=3 for each group) was recorded and plotted daily until experimental endpoint (day 14 following treatment). P<0.05 comparing survivals in WT-CVB3 group with those in PBS, miR-CVB3-2.1, or miR-CVB3-2.2 group by log-rank test (FIG. 6B). Hematoxylin and eosin (H&E) staining was performed on the heart and pancreas collected at day 4 post-treatment from a different cohort of mice (FIG. 6C) and pathological scores (mean±SD, n=3 mice) were evaluated based on the H&E staining (FIG. 6D). Immunostaining was conducted to assess viral protein VP1 expression in mouse heart and pancreas harvested on day 4 post-infection (FIG. 6E) and relative optical density of VP1 (mean±SD, n=3 mice) on day 4 and 10/14 (WT-CVB3 and miR-CVB3, respectively) post-treatment based on immunostaining images was also quantified (FIG. 6F). *, p<0.05; #, p<0.01; &, p<0.005; $, p<0.001 compared to WT-CVB3. ns, not significant. PI, post-infection. Scale bar=100 μM.

Mice treated with WT-CVB3 started to lose weight at day 4 and all died or had to be euthanized due to severe toxicity at day 9-10 after treatment. In contrast, mice injected with miR-CVB3-2.1 or miR-CVB3-2.2 exhibited a similar body weight increase as sham-infected mice and all mice survived the 2-week monitoring period. Pathological quantitation of the H&E staining revealed extensive tissue damage and inflammatory infiltration in the heart and pancreas of mice at day 4 following WT-CVB3 treatment. However, cardiac and pancreatic pathogenesis was markedly reduced to a barely detectable level in mice treated with either miR-CVB3-2.1 or miR-CVB3-2.2. Likewise, immunostaining showed that viral protein VP1 expression was drastically decreased in the heart and pancreas of mice treated with miR-CVB3-2.1 or miR-CVB3-2.2 in comparison with WT-CVB3-treated mice at day 4 post-treatment. As expected, the level of VP1 was significantly lower on day 10 (for WT-CVB3) or 14 (for miR-CVB3) than that on day 4 as a result of viral clearance. Collectively, the results indicate that the new miR-CVB3s have a safe profile when used in immunocompetent mice for the timeframe examined in this study.

### Example 7

Evaluation of Anti-Tumor Efficacy and Safety of New miR-CVB3s in a Xenograft Mouse Model

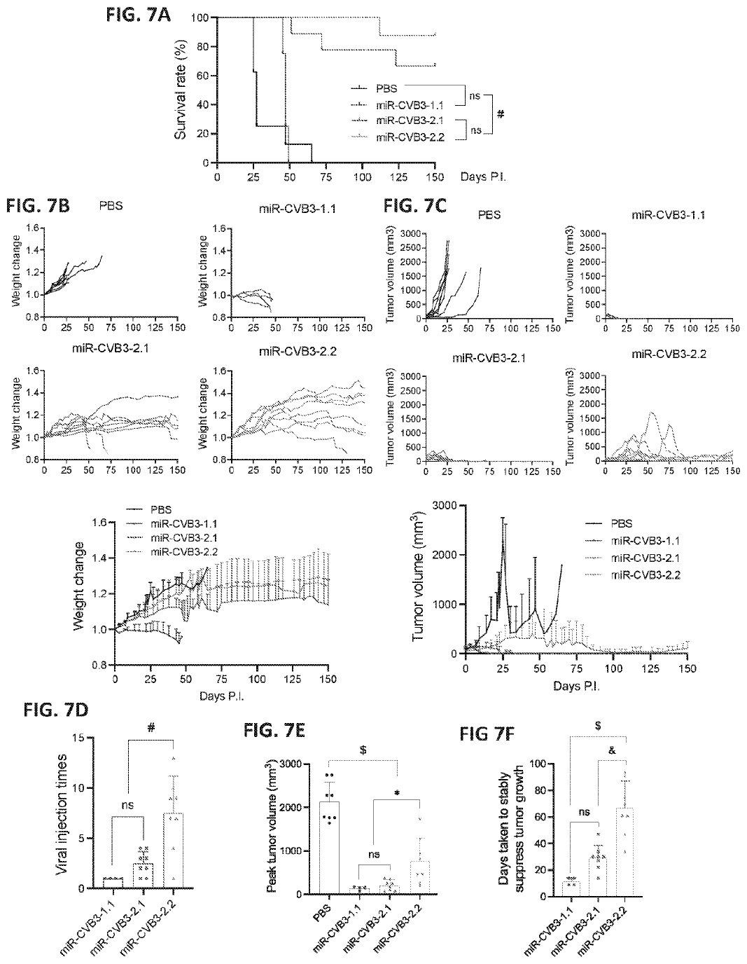

Immunocompromised non-obese diabetic-severe combined immune deficiency (NOD-SCID) mice bearing H526-derived SCLC xenografts were injected intraperitoneally with PBS, miR-CVB3-1.1, miR-CVB3-2.1, or miR-CVB3-2.2 at a dose of 1×10⁶ PFU weekly until the tumor growth was stably repressed (<100 mm3 for two weeks). Kaplan-Meier analysis revealed that the survival rates for mice treated with miR-CVB3-2.1 or miR-CVB3-2.2 at day 150 (experimental endpoint) were 70% and 90% respectively, while mice inoculated with miR-CVB3-1.1 all died or were euthanized because of severe viral toxicities prior to day 48 following treatment (FIG. 7A). In the PBS-treated group, mice were euthanized over the time-course due to surpassed tumor size threshold as per the animal care guideline. FIG. 7B depicts the changes in body weight (from individual mice or average) in each treatment group. Body weight was measured twice every week and normalized to those on day 0 (mean ±SD). Mice that underwent significant loss in body weight were euthanized.

The ability of miR-CVB3s to suppress xenograft tumors was assessed by tumor growth curve, which revealed a marked reduction in tumor volume upon treatment with different miR-CVB3s as compared to PBS control (FIG. 7C). Tumor volume was measured twice weekly until experimental endpoint (mean±SD). Viral injection times needed for steady and significant tumor suppression were also analyzed. FIG. 7D shows that the average viral injection numbers to achieve tumor suppression were comparable between miR-CVB3-1.1 and miR-CVB3-2.1, while being significantly higher for the miR-CVB3-2.2 group. The peak tumor volumes and days taken to repress the tumor growth were also examined. The anti-tumor ability of miR-CVB3-1.1 and miR-CVB3-2.1 were significantly more profound than that of miR-CVB3-2.2 (FIGS. 7E and 7F). *, p<0.05; #, p<0.01; &, p<0.005; $, p<0.001. ns, not significant. PI, post-infection.

Taken together, the results indicate that both miR-CVB3-2.1 and miR-CVB3-2.2 are safe in mice and retain robust oncolytic capability with the former displaying a greater anti-tumor activity than the latter.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

It is also to be understood that as used herein and in the appended claims, the singular forms “a,” “an,” and “the” include plural reference unless the context clearly dictates otherwise, the term “X and/or Y” means “X” or “Y” or both “X” and “Y,” and the letter “s” following a noun designates both the plural and singular forms of that noun. In addition, where features or aspects of the invention are described in terms of Markush groups, it is intended, and those skilled in the art will recognize, that the invention embraces and is also thereby described in terms of any individual member and any subgroup of members of the Markush group, and Applicants reserve the right to revise the application or claims to refer specifically to any individual member or any subgroup of members of the Markush group.

It is to be understood that the terminology used herein is for the purpose of describing specific embodiments only and is not intended to be limiting. It is further to be understood that unless specifically defined herein, the terminology used herein is to be given its traditional meaning as known in the relevant art.

Reference throughout this specification to “one embodiment” or “an embodiment” and variations thereof means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents, i.e., one or more, unless the content and context clearly dictates otherwise. It should also be noted that the conjunctive terms, “and” and “or” are generally employed in the broadest sense to include “and/or” unless the content and context clearly dictates inclusivity or exclusivity as the case may be. Thus, the use of the alternative (e.g., “or”) should be understood to mean either one, both, or any combination thereof of the alternatives. In addition, the composition of “and” and “or” when recited herein as “and/or” is intended to encompass an embodiment that includes all of the associated items or ideas and one or more other alternative embodiments that include fewer than all of the associated items or ideas.

Unless the context requires otherwise, throughout the specification and claims that follow, the word “comprise” and synonyms and variants thereof such as “have” and “include,” as well as variations thereof such as “comprises” and “comprising” are to be construed in an open, inclusive sense, e.g., “including, but not limited to.” The term “consisting essentially of” limits the scope of a claim to the specified materials or steps, or to those that do not materially affect the basic and novel characteristics of the claimed invention.

Any headings used within this document are only being utilized to expedite its review by the reader and should not be construed as limiting the invention or claims in any manner. Thus, the headings and Abstract of the Disclosure provided herein are for convenience only and do not interpret the scope or meaning of the embodiments.

Where a range of values is provided herein, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

For example, any concentration range, percentage range, ratio range, or integer range provided herein is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means±20% of the indicated range, value, or structure, unless otherwise indicated.

All of the U.S. patents, U.S. patent application publications, U.S. patent applications, foreign patents, foreign patent applications and non-patent publications referred to in this specification and/or listed in the Application Data Sheet, are incorporated herein by reference, in their entirety. Such documents may be incorporated by reference for the purpose of describing and disclosing, for example, materials and methodologies described in the publications, which might be used in connection with the presently described invention. The publications discussed above and throughout the text are provided solely for their disclosure prior to the filing date of the present application. Nothing herein is to be construed as an admission that the inventors are not entitled to antedate any referenced publication by virtue of prior invention.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

Furthermore, the written description portion of this patent includes all claims. Furthermore, all claims, including all original claims as well as all claims from any and all priority documents, are hereby incorporated by reference in their entirety into the written description portion of the specification, and Applicants reserve the right to physically incorporate into the written description or any other portion of the application, any and all such claims. Thus, for example, under no circumstances may the patent be interpreted as allegedly not providing a written description for a claim on the assertion that the precise wording of the claim is not set forth in haec verba in written description portion of the patent.

The claims will be interpreted according to law. However, and notwithstanding the alleged or perceived ease or difficulty of interpreting any claim or portion thereof, under no circumstances may any adjustment or amendment of a claim or any portion thereof during prosecution of the application or applications leading to this patent be interpreted as having forfeited any right to any and all equivalents thereof that do not form a part of the prior art.

Other nonlimiting embodiments are within the following claims. The patent may not be interpreted to be limited to the specific examples or nonlimiting embodiments or methods specifically and/or expressly disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

What is claimed is:

1. A replicating oncolytic virus vector comprising a modified Enterovirus genome, wherein the modified Enterovirus genome comprises one or more copies of one or more miRNA target sequences inserted into the UTR region via substitution and/or in-frame into the coding region of the Enterovirus genome.

2. The replicating oncolytic virus vector of claim 1, wherein said Enterovirus is a Coxsackievirus.

3. The replicating oncolytic virus vector of claim 2, wherein the Coxsackievirus is Coxsackievirus A or B.

4. The replicating oncolytic virus vector of claim 1, wherein the coding region is the region between the genes encoding proteins selected from the group consisting of VP4 and VP2, VP2 and VP3, VP3 and VP1, VP1 and 2A, 2A and 2B, 2B and 2C, 2C and 3A, 3A and 3B, 3B and 3C, and 3C and 3D.

5. The replicating oncolytic virus vector of claim 1, wherein the one or more copies of the one or more miRNA target sequences comprises one or more copies of two or more different miRNA target sequences.

6. The replicating oncolytic virus vector of claim 1, wherein spacers of 1 to 50 base pairs in size are inserted between the one or more miRNA target sequences.

7. The replicating oncolytic virus vector of claim 1 wherein the one or more copies of the one or more miRNA target sequences are targeted by miRNAs enriched in cardiac or pancreatic tissues.

8. The replicating oncolytic virus vector of claim 1, wherein the one or more different miRNA target sequences are targeted by an miRNA selected the group consisting of miR-1, miR-7, miR-30c, miR-124, miR-124*, miR-127, miR-128, miR-129, miR-129*, miR-133, miR-135b, miR-136, miR-136*, miR-137, miR-139-5p, miR-143, miR-154, miR-184, miR-188, miR-204, miR-208, miR-216, miR217, miR-299, miR-300-3p, miR-300-5p, miR-323, miR-329, miR-337, miR-335, miR-341, miR-369-3p, miR-369-5p, miR-375, miR-376a, miR-376a*, miR-376b-3p, miR-376b-5p, miR-376c, miR-377, miR-379, miR-379*, miR-382, miR-382*, miR-409-5p, miR-410, miR-411, miR-431, miR-433, miR-434, miR-451, miR-466b, miR-485, miR-495, miR-499, miR-539, miR-541, miR-543*, miR-551b, miR-758, and miR-873.

9. The replicating oncolytic virus vector of claim 8, wherein the two or more different miRNA target sequences comprise target sequences for miR-1, miR-133, miR-216, and miR-375.

10. The replicating oncolytic virus vector of claim 9, comprising one, two, three, four, five, or six copies of the target sequence for miR-1, miR-133, miR-216, and mR-375.

11. The replicating oncolytic virus vector of claim 1, wherein one or more copies of the one or more miRNA target sequences is in a forward orientation and one or more copies of the one or more miRNA target sequences is in a reverse orientation.

12. The replicating oncolytic virus vector of claim 1, wherein the modified Enterovirus genome comprises at least one nucleic acid encoding a non-viral protein selected from the group consisting of immunostimulatory factors, antibodies, and checkpoint blocking peptides, wherein the at least one nucleic acid is operably linked to a suitable tumor-specific regulatory region.

13. The replicating oncolytic virus vector of claim 12, wherein the non-viral protein is selected from the group consisting of IL12, IL15, IL15 receptor alpha subunit, OX40L, CD73, and a checkpoint inhibitor.

14. A method for lysing tumor cells, comprising providing an effective amount of a first replicating oncolytic virus vector of any of claims 1 to 13 to tumor cells.

15. The method of claim 14, wherein the tumor cells comprise lung cancer cells.

16. The method of claim 14, wherein the tumor cells comprise pancreatic cancer cells, liver cancer cells, or breast cancer cells.

17. A therapeutic composition comprising at least one replicating oncolytic virus vector of any of the above claims and a pharmaceutically acceptable carrier.

18. A method for treating cancer in a subject suffering therefrom, comprising the step of administering a composition comprising a therapeutically effective amount of the composition of claim 17.

19. The method of claim 18, wherein the cancer is non-small-cell lung cancer (NSCLC) associated with KRAS mutations, small-cell lung cancer (SCLC) commonly linked to TP53 and Rb mutations, or pancreatic cancer.

20. The method of claim 18, wherein the administration is intravenous (IV) administration, intraperitoneal (IP) administration, or intratumoral (IT) administration.
